(19) United States
(12) Patent Application Publication
(10) Pub. No.: US 2025/0026839 A1
(43) Pub. Date: Jan. 23, 2025

(54) Identification of CLEC-1 Ligand and Uses Thereof

(71) Applicants: OSE IMMUNOTHERAPEUTICS, Nantes (FR); NANTES UNIVERSITE, Nantes (FR); INSTITUT NATIONAL DE LA SANTE ET DE LA RECHERCHE MEDICALE, Paris Cedex (FR)

(72) Inventors: Vanessa Gauttier, Reze (FR); Caroline Mary, Sainte-Pazanne (FR); Nicolas Poirier, Grandchamps des Fontaines (FR); Emmanuelle Wilhelm, Fay de Bretagne (FR); Géraldine Teppaz, Saint Herblain (FR); Elise Chiffoleau, Nantes (FR)

(21) Appl. No.: 18/707,922

(22) PCT Filed: Nov. 9, 2022
(86) PCT No.: PCT/EP2022/081332

§ 371 Date: May 7, 2024

(30) Foreign Application Priority Data
Nov. 9, 2021 (EP) 21306571.7

(51) Int. Cl. C07K 16/28; C07K 14/47

(52) U.S. Cl. CPC C07K 16/2851; C07K 14/4726; C07K 2317/73; C07K 2317/76; C07K 2319/30

(57) ABSTRACT

The invention relates to the use of compounds that reduce the signaling pathway induced by CLEC-1/TRIM21 interaction, in particular compounds that antagonize the interaction, in particular the binding between CLEC-1 and TRIM21, the use of the identified ligand TRIM21 for designing treatment in a patient, in the identification of cells susceptible to interact with cells expressing CLEC-1, and their uses in therapy.

### FIELD OF THE INVENTION

The invention follows the identification of a ligand of CLEC-1 protein, namely TRIM21. The invention relates to the use of compounds that reduce the signaling pathway induced by CLEC-1/TRIM21 interaction, in particular compounds that antagonize the interaction, in particular the binding between CLEC-1 and TRIM21, the use of the identified ligand TRIM21 for designing treatment in a patient, in the identification of cells susceptible to interact with cells expressing CLEC-1, and their uses in therapy.

The invention also concerns a method enabling detection of the interaction of CLEC-1 with TRIM21 to assess antagonist capability of compounds towards the CLEC-1/TRIM21 signaling pathway, or to screen compounds capable of binding to CLEC-1 or to TRIM21, in particular capable of competing with the binding between CLEC-1 and TRIM21.

### BACKGROUND OF THE INVENTION

One of the issues in the treatment of cancer is to destroy tumor cells without damaging healthy tissue. In recent years, a major issue in the treatment of cancer is to target tumor cells without damaging healthy tissue. Anti-tumoral immunotherapies gained an increasing interest in the past decade as they redirect the patient's immune system to destroy cancer cells. It has been clearly demonstrated that tumor cells exploit immune checkpoints to escape immune defenses. The critical roles of these inhibitory checkpoints in obstructing anti-tumor immunity have been illustrated by the remarkable success of antibody-driven blockade of T-cell immune checkpoints. However, a significant proportion of patients do not respond or develop resistance to these checkpoint inhibitor (CKI) therapies, generally after classical chemotherapy failure. Effective novel treatments are needed for these patients, and myeloid cells represent promising therapeutic target. Indeed, myeloid cells are the most abundant tumor infiltrating immune cells. C-type lectins (CLEC) expressed on immune cells are sensors of the environment and immune response modulators. CLEC-1 represents a potential therapeutic target to boost myeloid cells and anti-tumor response. To better decipher CLEC-1 function and develop compounds aimed at modulating its activity, there is a need of identifying its ligands, especially in the tumor environment context.

CLEC-1 (also designated CLEC-1A, or CLEC1, or CLEC1A) is a C-type lectin-like receptor, i.e., C-type lectin-like receptor-1, that is in particular expressed in mammal species, more particularly in human. More precisely, CLEC-1 belongs to the DECTIN-1 cluster of C type-lectin like receptors (CTLRs) that also includes CLEC-2, DECTIN-1 (CLEC7-A), CLEC-9A, MICL, MAH and LOX-1 (Colonna M, Samaridis J, Angman L. Molecular characterization of two novel C-type lectin-like receptors, one of which is selectively expressed in human dendritic cells. Eur J Immunol. 2000; 30(2):697-704). Functionally, C-type lectin receptors (CLRs) are a large family of transmembrane and soluble receptors receptors that contain one or more carbohydrate-recognition domain able to recognize a wide variety of glycans on pathogens or on self-proteins. For these receptors, glycan recognition is dependent from Ca²⁺. Many related-CLRs are nonetheless able to recognize carbohydrates but independently of Ca²⁺; these receptors are referred to C-type lectin-like receptors (CTLRs). These receptors are of particular interest for their role in coupling both innate and adaptive immunity. CTLRs are expressed mostly by cells of myeloid lineage such as monocytes, macrophages, dendritic cells (DCs), and neutrophils. CTLRs not only serve as antigen-uptake receptors for internalization and presentation to T cells but also trigger multiple signaling pathways leading to NF-κB, type I interferon (IFN), and/or inflammasome activation. By their capacity to present antigen and ensure the balance between cellular activation and suppression, CTLRs have emerged as challenging pharmacological targets to treat a wide variety of diseases including cancers, autoimmune diseases or allergy. CTLR modulation seems to represent a promising strategy for disease management although attempts at identifying ligands as well as efforts to elucidate their role in immunity remain incomplete to date.

CLEC-1 is known to be expressed on myeloid and endothelial cells. CLEC-1 has been described as a receptor that may be up-regulated by immunomodulatory mediators and that may moderate T cell activation (Thebault P. et al The C-Type Lectin-Like Receptor CLEC-1, Expressed by Myeloid Cells and Endothelial Cells, Is Up-Regulated by Immunoregulatory Mediators and Moderates T Cell Activation. J Immunol 2009; 183:3099-3108). The present inventors showed that CLEC-1 is expressed at the cell-surface by conventional DCs (cDCs) and by small subsets of monocytes and DCs in human blood and is enhanced by the immunosuppressive cytokine TGFβ (see international application No. WO2018073440). They demonstrated in both rodent and human that CLEC-1 acts as an inhibitory receptor in myeloid cells and prevent IL12p40 expression and downstream Th1 and Th17 in vivo responses (Lopez-Robles M D et al., Cell-surface C-type lectin-like receptor CLEC-1 dampens dendritic cell activation and downstream Th17 responses Blood Adv. 2017; Mar. 22; 1(9) 557-568).

These properties provide interest to consider CLEC-1 in the design of additional means for the treatment of health conditions that involve the immune response of the patient, in particular to develop new treatment against cancer. In particular, these results call for additional research in order to identify the expression compound(s) that may be endogenous ligand(s) of the receptor. Endogenous ligand refers to a mammalian ligand, and in particular to a human ligand, by contrast to exogeneous ligand, issued from a different species than a mammalian. Indeed, an exogenous ligand for CLEC-1 has been identified on a pathogenic organism, i.e., Aspergillus Fumigatus, said ligand being the naphthalene-diol unit of 1,8-dihydroxynaphthalene (DHN)-melanin (Stappers M H T et al Recognition of DHN-melanin by a C-type lectin receptor is required for immunity to Aspergillus Nature 2018 Mar. 15; 555(7696):382-386). This exogenous ligand has been shown to be present in mice or human in conditions of infection by said pathogenic fungus.

There was a need for identification of CLEC-1 ligand, in particular endogenous ligand, and for determination of CLEC-1 binding thereto, in order to identify pathways involved in modulating an immune response or used to design/identify compounds for modulating an immune response.

### DESCRIPTION OF THE INVENTION

The present invention relies on the identification of a ligand of CLEC-1 endogenous of human cells. The present inventors have identified that TRIM21 (tripartite motif-containing protein 21) is a ligand of CLEC-1, at least in human. The inventors have been able to observe that TRIM21 and CLEC-1 are able to interact (i.e. bind to each other) in vitro. Starting from a lysate of RAJI cell line, the inventors were able to purify and identify a protein which is able to interact (i.e. bind) with a functional region of CLEC-1 (Fc-CLEC1). The inventors also observed that several anti-TRIM21 antibodies and anti-CLEC-1 antibodies are able to inhibit the interaction between TRIM21 and CLEC-1, and thus are antagonists of the binding between CLEC-1 and TRIM21. In particular, the inventors observed that recombinant or fusion molecules including in particular recombinant or fusion polypeptides, wherein said molecule comprises the extracellular domain of CLEC-1, in particular extracellular domain of human CLEC-1 (as illustrated herein by Fc-CLEC-1), are able to bind to TRIM21, and that this binding was disrupted by several anti-TRIM21 and anti-CLEC-1 antibodies.

As will be detailed further in the application, the inventors provide evidence that the antagonism of the CLEC1/TRIM21 signaling pathway leads notably to increase the phagocytosis capability of myeloid cells, in particular of macrophages and/or dendritic cells, particularly it leads to an increase phagocytosis of TRIM21-positive cells by myeloid cells, in particular an increase of phagocytosis of tumor cells and/or secondary necrotic cells, more particularly of TRIM21-positive tumor cells and/or secondary necrotic cells, by dendritic cells and/or macrophages.

The tripartite motif-containing (TRIM) proteins all contain a structurally conserved N-terminal with Really Interesting New Gene (RING) motif followed by one or two zinc-finger domains called B-boxes and a coiled-coil (CC) domain. Like any other family members, TRIM21 is an E3 ligase via its RING domain. TRIM21, as a member of the TRIM family, is composed of five domains: RING, B-box, coiled-coil, PRY and SPRY (these two last domains are often associated within the PRY-SPRY domain. Human TRIM21 may have the amino acid sequence of SEQ ID No. 2, and the RING, B-box, coiled-coil, PRY-SPRY domains may be localized between amino acid residues 16 and 55, 92 and 123, 128 and 238, 268 and 465 respectively. TRIM21 regulates innate immune signaling through its E3 ligase function. It is an autoantigen related to autoimmune diseases, such as systemic lupus erythematosus and Sjögren's syndrome. Previous studies (Brauner et al, Journal of Internal Medecine, Vol. 278(3), September 2015, pages 323-332) suggest that TRIM21 may play a role in tumor development and recent studies have explored the relationships between TRIM21 expression and tumor prognosis (Ding, Q., He, D., He, K. et al. Downregulation of TRIM21 contributes to hepatocellular carcinoma carcinogenesis and indicates poor prognosis of cancers. Tumor Biol. 36, 8761-8772 (2015). https://doi.org/10.1007/s13277-015-3572-2). High and low-level expressions of TRIM21 are usually associated with good and poor prognosis, respectively. Following the discovery in the present invention that TRIM21 is a ligand of CLEC-1, and due to the involvement of CLEC-1 in immune response, TRIM21 could be considered as a target for development of a novel therapeutic agent, a novel diagnostic tool, a prognostic factor, and therapeutic factor, in particular in the treatment of cancers or autoimmune diseases, as well as the TRIM21/CLEC1 pathway, in particular interaction.

In a first aspect, the invention relates to a compound that inhibits the C-type lectin-like receptor-1 (CLEC-1)/tripartite motif-containing protein 21 (TRIM21) signaling pathway, for its use as a medicament, particularly in the treatment of a subject, in particular a human subject, who has a disease selected from cancer listed in the present description, in particular cancers with TRIM21-positive tumor cells, such as breast cancer, hepatocellular carcinoma, lymphoma, more particularly B-cell lymphoma, colon cancer, thyroid cancer, liver cancer, renal cancer, melanoma, colorectal cancer, nasopharyngeal carcinoma, pancreatic cancer, a chronic infection, a sepsis, an infection, a viral infection, in particular by a Coxsackievirus or by an encephalitis virus, more particularly by Coxsackievirus B3 or Japanese encephalitis virus, a fungi infection, a cardiovascular disease, an auto-immune disease, in particular Sjogren's syndrome or systemic lupus erythematosus or systemic sclerosis, an inflammatory disease.

In another aspect, the invention relates to a compound that is an antagonist of the binding between CLEC-1 and TRIM21, in particular between human CLEC-1 and human TRIM21, which binds to TRIM21, for its use as a medicament, in particular for the treatment of a subject, in particular a human subject, who has a disease selected from cancer listed in the present description, in particular cancers with TRIM21-positive tumor cells, such as breast cancer, hepatocellular carcinoma, lymphoma, more particularly B-cell lymphoma, colon cancer, thyroid cancer, liver cancer, renal cancer, melanoma, colorectal cancer, nasopharyngeal carcinoma, pancreatic cancer, a chronic infection, a sepsis, an infection, in particular by a Coxsackievirus or by an encephalitis virus, more particularly by Coxsackievirus B3 or Japanese encephalitis virus, a cardiovascular disease, an auto-immune disease, in particular Sjogren's syndrome or systemic lupus erythematosus or systemic sclerosis, an inflammatory disease. In a preferred embodiment, the patient has a cancer, a sepsis or a chronic infection.

In another aspect, the invention also relates to the use of a compound that inhibits the C-type lectin-like receptor-1 (CLEC-1)/tripartite motif-containing protein 21 (TRIM21) signaling pathway, for increasing the phagocytosis of cells, in particular TRIM21-positive cells, in particular tumor cells and/or secondary necrotic cells, more particularly TRIM-21 positive tumor cells and/or secondary necrotic cells, by myeloid cells, in particular by macrophages and/or dendritic cells. The compound that can be used includes antibodies, antigen-binding fragment thereof, antigen-binding antibody mimetics, a peptides or polypeptides which are a functional equivalents of TRIM21 or CLEC-1, like but not limited to Fc-CLEC-1, in particular Fc-CLEC-1 comprising the amino acid residues set forth in SEQ ID No. 3, a nucleic acid molecule, in particular an oligonucleotide molecule, more particularly a RNA molecule, a siRNA molecule or a miRNA molecule, which reduces the expression of TRIM21 and/or CLEC-1.

In another aspect, the invention also relates to a method in which the interaction between CLEC-1 and TRIM 21 is used for assessing the likelihood of effectiveness of a treatment with an anti-cancer agent and/or with an antagonist anti-human CLEC-1 antibody or antigen-binding fragment thereof, or with an antagonist anti-TRIM21 antibody or antigen-binding fragment thereof; for screening antagonist compounds; for treating diseases.

### Definitions

As used herein, the terms “CLEC-1” and “CLEC-1A” relates to a CLEC-1A protein from a mammal species, preferably a human CLEC-1 or CLEC-1A. A reference sequence of the human CLEC-1A receptor corresponds to the sequence associated to the Accession number Q8NC01 Uniprot. Preferably, the term “human CLEC-1” refers to the protein of amino acid sequence referenced by the Q8NC01 Uniprot accession number and encoded by CLEC-1 gene referenced by the 51267 NCBI accession number. In the present description, the terms CLEC-1A, CLEC-1, CLECA, CLEC-1, Clec1, Clec-1, CLEC-A1 and Clec-1A are used interchangeably and all designate the CLEC1 receptor of a mammal corresponding to human CLEC-1A receptor characterized by the amino acid sequence associated to the Accession number Q8NC01 Uniprot, an orthologue protein thereof, or a homologous protein thereof.

Preferably, the term “human CLEC-1” refers to the protein of amino acid sequence referenced by the Q8NC01 Uniprot accession number and encoded by CLEC-1 gene referenced by the 51267 NCBI accession number. CLEC-1 may be characterized by the amino acid sequence set forth in SEQ ID No. 1 In a particular embodiment, the amino acid sequence of the extracellular domain of human CLEC-1 comprises or consists in the sequence of QYYQLSNTGQDTISQMEERLGNTSQELQSLQVQNIKLAGSLQHVAEKLCRELYNKA GAHRCSPCTEQWKWHGDNCYQFYKDSKSWEDCKYFCLSENSTMLKINKQEDLEF AASQSYSEFFYSYWTGLLRPDSGKAWLWMDGTPFTSELFHIIIDVTSPRSRDCVAIL NGMIFSKDCKELKRCVCERRAGMVKPESLHVPPETLGEGD (SEQ ID No. 3).

As used herein a “functional equivalent of CLEC-1” in particular “functional equivalent of human CLEC-1” is a polypeptide, a peptide, an oligopeptide, a protein, in particular comprising at least a fragment (or portion) of the extracellular domain of CLEC-1 and possibly comprising the extracellular domain of CLEC-1 with the proviso that it is not the entire CLEC-1 polypeptide. Such fragment of the extracellular domain of CLEC-1 or whole extracellular domain of CLEC-1 may be combined with another molecule, like a peptide or a fragment of another protein like an antibody, which stabilizes the structure of the extra-cellular domain of CLEC-1 or of the fragment thereof, leading to the provision of a fusion protein comprising at least a fragment of the extra-cellular domain of CLEC-1. Such a functional equivalent of CLEC-1, in particular of human CLEC-1 may alternatively be a polypeptide, a peptide, an oligopeptide, a protein whose amino acid sequence has at least 80%, in particular at least 90% identity with the aligned amino acid sequence set forth in SEQ ID No. 3 (so-called variant of SEQ ID No. 3).

In particular, the functional equivalent of CLEC-1 comprises an extracellular domain of CLEC-1 or comprises a sequence which has an identity of at least 80% in particular more than 80%, in particular at least 85% in particular more than 85%, at least 90% in particular more than 90%, at least 95% in particular more than 95%, with the extracellular domain of CLEC-1, especially of sequence set forth in SEQ ID NO: 3.

In particular the functional equivalent of CLEC-1 is a functional equivalent of human CLEC-1 and comprises an extracellular domain of human CLEC-1 or comprises a sequence which has an identity of at least 80% in particular more than 80%, in particular at least 85% in particular more than 85%, at least 90% in particular more than 90%, at least 95% in particular more than 95% with the extracellular domain of human CLEC-1, more particularly having at least 80% (in particular at least or more than 85%, at least or more than 90%, at least or more than 95%) identity with the amino acid sequence set forth in SEQ ID NO: 3.

The functional equivalent of CLEC-1, in particular of human CLEC-1 may for example comprise the fragment of the extra-cellular domain of CLEC-1 or the whole extra-cellular domain or a variant thereof having at least or more than 80%, in particular at least or more than 90%, in particular at least or more than 95% identity with the aligned amino acid sequence in the sequence of SEQ ID No. 3 and furthermore comprises a linker peptide, a tag, a Fc portion of an antibody. Fc-CLEC-1 may for example comprise the amino acid sequence set forth in SEQ ID No. 3. This functional equivalent of CLEC-1 may for example be a fusion polypeptide. In a particular embodiment, the compound of the invention is a polypeptide that is a functional equivalent of CLEC-1, in particular of human CLEC-1, fused to an immunoglobulin constant domain.

According to a particular embodiment of the invention, a fusion molecule is provided that features a functional equivalent of CLEC-1 as defined herein. Accordingly the extracellular domain of CLEC-1 may be fused to a tag (such as a tag comprising an amino acid sequence) or a moiety suitable to stabilize the extracellular domain of CLEC-1 when used to target the ligand and/or an immunoglobulin constant domain (Fc region), in particular a constant domain of a human immunoglobulin to form a fusion molecule that can possess many of the valuable chemical and biological properties of human antibodies and/or a linker as defined herein.

In a particular embodiment of the fusion molecule, the Fc fragment of a human immunoglobulin contained in the Fc-CLEC-1 fusion molecule is the Fc fragment of a human IgG1 and the Fc fragment is fused N-terminal to the extracellular domain of the mammalian CLEC-1, in particular of the human CLEC-1.

As used herein, the term “TRIM21” has its general meaning in the art and refers to tripartite motif-containing protein 21, particularly from a mammal species, more particularly a human TRIM21.

Preferably, the term “human TRIM21” refers to the protein of amino acid sequence referenced by the P19474 Uniprot accession number and encoded by TRIM21 gene referenced by the Gene ID 6737 (NCBI accession number). In a particular embodiment, TRIM21 has the amino acid sequence set forth in SEQ ID No. 2. In particular, TRIM21 is a ligand of CLEC-1 and is capable of binding to CLEC-1 (for example when the cell expressing TRIM21 is permeabilized, like permeabilized RAJI cells, or some kind of tumor cells), in particular to the extracellular domain of CLEC-1, in particular to CLEC-1 of SEQ ID No. 1.

As used herein, the terms “TRIM21 positive cells”, “TRIM21-positive tumors” and “TRIM21-positive tumor cells” refers to cells or group of cells that express the TRIM21 protein, either by constitutive expression (e.g. due to the normal expression of the gene encoding the TRIM21 protein by the cell), or by induced expression (e.g. due to external stimulation, or by cell ongoing into stressful condition, or ongoing into apoptotic pathway, etc.).

### Antagonists of the CLEC-1/TRIM21 Signaling Pathway

In a first aspect, the invention relates to a compound which is an antagonist of the CLEC-1/TRIM21 signaling pathway, i.e. which inhibits the CLEC-1/TRIM21 signaling pathway. In the present description, the expression “compound which is an antagonist of the CLEC-1/TRIM21 signaling pathway” and the term “antagonist compound” are used interchangeably. The CLEC-1/TRIM21 signaling pathway includes any compound involved in the chemical reactions in a cell to control a cell function following the interaction between CLEC-1 and TRIM21. A cell receives a signal from its environment when CLEC-1 and TRIM21 interact, the CLEC-1 compound or the TRIM21 compound being expressed by the cell. After the first molecule in the pathway receives a signal, it activates another molecule. This process is repeated through the entire signaling pathway until the last molecule is activated and the cell function is carried out. An antagonist of the CLEC-1/TRIM21 signaling pathway, i.e. a compound that inhibits the CLEC-1/TRIM21 signaling pathway, may correspond to one of the following molecule:
a molecule that binds, especially specifically binds, to TRIM21 or to CLEC1 and antagonize the binding between the TRIM21 and CLEC-1; or
a compound that reduces the expression of TRIM21 or CLEC1 within a cell, like nucleic acid molecule that can inhibit the expression or the translation into functional protein of TRIM21 or CLEC1, like but not limited to oligonucleotide molecules, more particularly a RNA molecule, a siRNA molecule or a miRNA molecule, that reduces the expression of functional TRIM21 or CLEC1 molecule within the cell; or
A functional equivalent of TRIM21 or of CLEC-1; or
a compound able to reduce or inhibit the capability of a compound involved in the chemical reaction induced by the binding between CLEC-1 and TRIM21 to activate or inhibit another different compound(s) involved in the same signaling pathway.

Other compound able to antagonize the signaling pathway activated or inhibited following the interaction between CLEC-1 and TRIM21 is contemplated for use according to the present invention.

The antagonist effect of a compound on the CLEC-1/TRIM 21 pathway may be assessed by measuring an increase (as compared to a negative control) of the phagocytosis of cells, in particular of TRIM21-positive cells, more particularly of tumor cells and/or secondary necrotic cells, more particularly of TRIM21-positive tumor cells and/or secondary necrotic cells, by myeloid cells, in particular by dendritic cells and/or macrophages.

In an embodiment, the invention relates to a compound which is an antagonist of the binding between CLEC-1 and TRIM21, in particular between human CLEC-1 and human TRIM21, which binds to TRIM21, especially binds specifically to TRIM21, for use as a medicament, particularly in the treatment of a subject, in particular a human subject, suffering from a disease selected from a cancer, in particular cancers with TRIM21-positive tumor cells, such as breast cancer, hepatocellular carcinoma, lymphoma, more particularly B-cell lymphoma, colon cancer, thyroid cancer, liver cancer, renal cancer, melanoma, colorectal cancer, nasopharyngeal carcinoma, pancreatic cancer, a chronic infection, a sepsis, an infection, in particular by a Coxsackievirus or by an encephalitis virus, more particularly by Coxsackievirus B3 or Japanese encephalitis virus, a cardiovascular disease, a cardiovascular disease, an auto-immune disease, in particular Sjogren's syndrome or systemic lupus erythematosus or systemic sclerosis, an inflammatory disease.

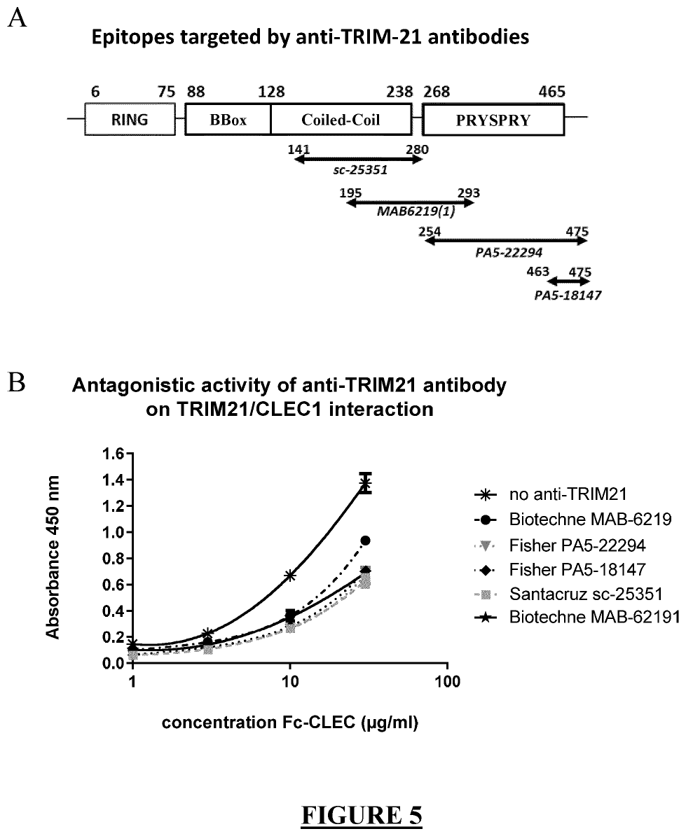

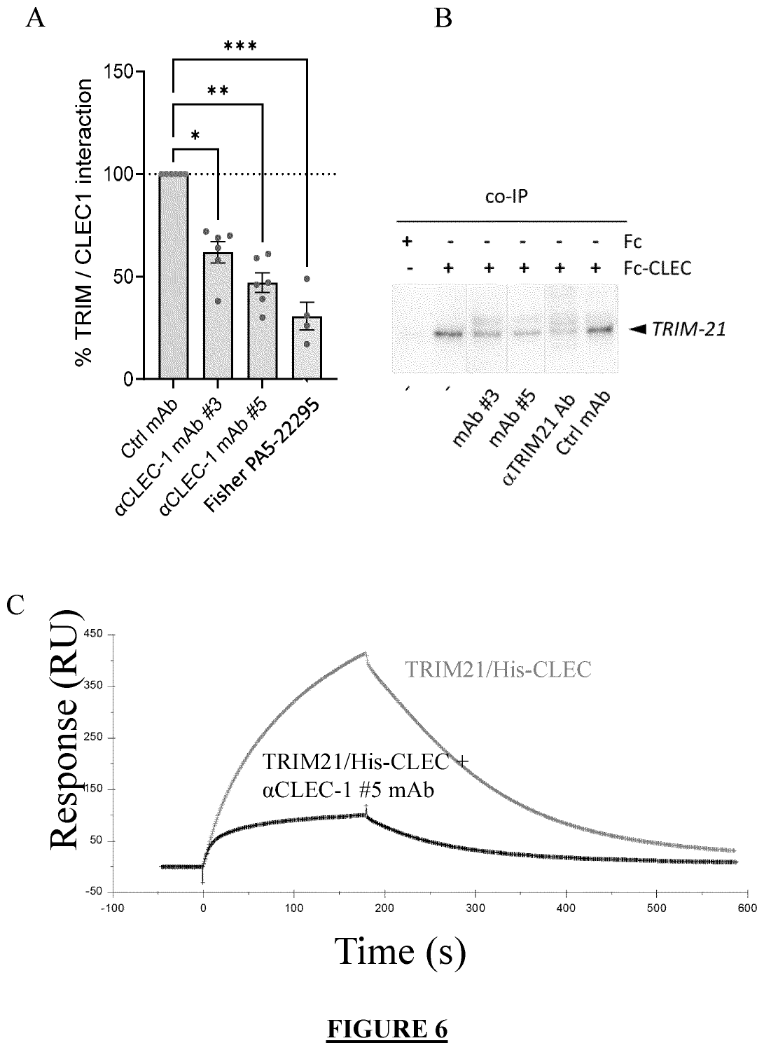

As used herein, a compound which is an antagonist of the binding between CLEC-1 and TRIM21 (also referenced in the present description as an “antagonist” or as an “antagonist compound”) may designate a compound which binds to TRIM21, especially specifically binds to TRIM21, and may be selected from the group consisting of an antibody, an antigen-binding fragment of an antibody, an antigen-binding antibody mimetic, a macromolecule comprising an antigen-binding fragment of an antibody or a full antibody, small organic compounds, a polypeptide, a peptide, an oligopeptide, a protein. Alternatively, the compound may correspond to a functional equivalent of TRIM21, in particular human TRIM21. Such a functional equivalent may be a polypeptide, a peptide, an oligopeptide, a protein, in particular comprising at least a fragment of the extra-cellular domain of TRIM21. Such fragment may be combined with another molecule, like a peptide or a fragment of another protein like an antibody, which stabilizes the structure of the fragment of the extra-cellular domain of TRIM21, leading to the provision of a fusion protein comprising at least a fragment of the extra-cellular domain of TRIM21. Such a fusion protein may for example comprise the fragment of the extra-cellular domain of TRIM21 and a linker peptide, a tag, a Fc portion of an antibody. In a particular embodiment, the compound may be a functional equivalent of CLEC-1, in particular human CLEC-1. Such a functional equivalent may be a polypeptide, a peptide, an oligopeptide, a protein, in particular comprising at least a fragment of the extra-cellular domain of CLEC-1 or may be a polypeptide, a peptide, an oligopeptide, a protein whose amino acid sequence has at least or more than 80%, in particular at least or more than 85%, in particular at least or more than 90% identity with the aligned amino acid sequence set forth in SEQ ID No. 3 (so-called variant). Such fragment or variant may be combined with another molecule, like a peptide or a fragment of another protein like an antibody, which stabilizes the structure of the fragment of the extra-cellular domain of CLEC-1, leading to the provision of a fusion protein comprising at least a fragment of the extra-cellular domain of CLEC-1. Such a fusion protein may for example comprise the fragment of the extra-cellular domain of CLEC-1 and a linker peptide, a tag, a Fc portion of an antibody; Fc-CLEC-1 may for example comprise the amino acid sequence set forth in SEQ ID No. 3. A compound which is an antagonist of the binding between CLEC-1 and TRIM21 is a compound able to reduce, inhibit or block the interaction between CLEC-1 and TRIM21, in vitro, in vivo and/or ex vivo. The antagonist effect on the binding between CLEC-1 and TRIM21 may be determined using methods as illustrated in the examples of the present application. In other words, a compound which is an antagonist and bind to TRIM21 does negatively interfere with the interaction between CLEC-1 and TRIM21, and consequently downregulates, reduces or inhibits a signaling pathway induced by the interaction between CLEC-1 and TRIM21. The presence or absence of such antagonist capability on the binding between CLEC-1 and TRIM21 may be assessed according to the examples of the invention, particularly in example 3, and illustrated on FIGS. 5 and 6, wherein a competition assay to measure the binding of TRIM21 to Fc-CLEC-1 protein in presence of an anti-TRIM21 antibody is disclosed. In a preferred embodiment, the antagonist compound of the invention does compete with CLEC-1 or Fc-CLEC-1, as disclosed herein, for the binding to TRIM21. The presence of an antagonist capability in a competition assay between TRIM21 on one hand and an antagonist compound of the invention and CLECL-1 or Fc-CLEC-1 on the other hand may be determined when, in presence of the antagonist compound of the invention, the binding of TRIM21 to CLEC-1 or Fc-CLEC-1 is lower than 90%, more preferably lower than 80%, still more preferably lower than 50% and most preferably lower than 20%, with respect to the binding of TRIM21 to CLEC-1 or Fc-CLEC-1, under the same experimental conditions but without the presence of the assessed antagonist of the invention. Alternatively, the antagonist capability towards the CLEC-1/TRIM21 binding may be determined according to the method illustrated in the examples of the invention. In particular, a compound binding to TRIM21 may be considered as an antagonist of the interaction between CLEC-1 and TRIM21, in particular between human CLEC-1 and human TRIM21, when it reduces the binding of the extra-cellular domain of CLEC-1, in particular when it reduces the binding of a fusion protein comprising the extracellular domain of human CLEC-1 receptor fused with a Fc fragment of a human immunoglobulin, in particular a human IgG, to TRIM21 as compared to the same binding experiment in absence of the assessed compound. A reduction is acknowledged when the binding is reduced by at least 1-log, more particularly at least 2-log and most preferably at least 3-log as compared to the control experiment.

In an aspect of the invention, the antagonist compound binds to TRIM21, especially specifically binds to TRIM21, in particular human TRIM21. In other words, the antagonist compound of the invention exhibits specific affinity for its target, TRIM21. In another aspect of the invention disclosed herein, the antagonist compound binds to CLEC-1, in particular human CLEC-1. In other words, the antagonist compound of the invention exhibits specific affinity for its target, CLEC-1. The terms “Specifically binding” and “specifically bind to” “specific affinity for” refer to the ability of the antagonist compound used according to the invention, in particular an antibody, antigen-binding fragment thereof, antigen-binding antibody mimetic or a modified antibody according to the invention, to bind to TRIM21 or respectively to CLEC-1 with an affinity of at least 1×10⁻⁶ M, 1×10⁻⁷ M, 1×10⁻⁸ M, 1×10⁻⁹ M, 1×10⁻¹⁰ M, 1×10⁻¹¹ M, 1×10⁻¹² M, or more, and/or to bind to TRIM21 or respectively to CLEC-1 with an affinity which is at least two-fold greater to its affinity for a non-specific target (e.g. another protein than TRIM21, like CLEC-1 or respectively another protein than CLEC-1, like TRIM21)). The affinity may be assessed according to various methods well known from those skilled in the art. These methods include but are not limited to biosensors such as Biacore analysis, Blitz analysis and Scatchard plot.

The antagonist compound may be an antibody, an antigen-binding fragment of an antibody, an antigen-binding antibody mimetic, a small organic compound, a peptide, a polypeptide, an oligopeptide, a protein, in particular a peptide or a polypeptide comprising a portion of TRIM21 amino acid sequence comprising contiguous amino acid residues as in the sequence of SEQ ID No. 2. The antagonist compound may also be an RNA molecule, in particular which reduce the expression of TRIM21 protein, like but not limited to Small Interfering RNA, double-stranded RNA, micro-RNA (miRNA), and having the capability to interfere with the RNA interference (RNAi) pathway. Such RNA molecule interferes with the expression of the gene encoding TRIM21 with complementary nucleotide sequences by degrading mRNA after transcription of the trim21 gene, thereby preventing its translation into the TRIM21 protein. Most preferred antagonist compounds are anti-TRIM21 antibodies.

In a particular embodiment of the invention, the compound is an antibody, an antigen-binding fragment of an antibody, or an antigen-binding antibody mimetic, which binds to an epitope sequence localized within the coiled-coil domain of TRIM21, in particular the coiled-coil domain localized between amino acid residues 128 and 238 of TRIM21 of SEQ ID No. 2 or the coiled-coil domain corresponding to amino acid residues of SEQ ID No. 4, and/or which binds to an epitope sequence localized within the PRY-SPRY domain of TRIM21, in particular the PRY-SPRY domain localized between amino acid residues 268 and 465 of TRIM21 of SEQ ID No. 2 or the PRY-SPRY domain corresponding to amino acid residues of SEQ ID No. 5. In a particular embodiment, the compound is an antibody, an antigen-binding fragment of an antibody, which binds to an epitope sequence localized between amino acid residues 141 and 280 of the TRIM21 of SEQ ID No. 2 or to an epitope localized within the amino acid sequence set forth in SEQ ID No. 7. In a particular embodiment, the compound is an antibody, an antigen-binding fragment of an antibody, which binds to an epitope sequence localized between amino acid residues 195 and 293 of the TRIM21 of SEQ ID No. 2 or to an epitope localized within the amino acid sequence set forth in SEQ ID No. 8. In a particular embodiment, the compound is an antibody, an antigen-binding fragment of an antibody, which binds to an epitope sequence localized between amino acid residues 254 and 475 of the TRIM21 of SEQ ID No. 2 or to an epitope localized within the amino acid sequence set forth in SEQ ID No. 6. In a particular embodiment, the compound is an antibody, an antigen-binding fragment of an antibody, which binds to an epitope sequence localized between amino acid residues 463 and 475 of the TRIM21 of SEQ ID No. 2 or to an epitope localized within the amino acid sequence set forth in SEQ ID No. 9.

In a particular embodiment of the invention, the compound is an antibody, an antigen-binding fragment thereof or an antigen-binding antibody mimetic, which binds to TRIM21, especially specifically binds to TRIM21, and which antagonizes the binding between CLEC1 and TRIM21, in particular between human CLEC-1 and human TRIM21, said antibody, antigen-binding fragment thereof or antigen-binding antibody mimetic competes with at least one competing antibody selected among the list consisting of anti-TRIM21 antibodies sc-25351, PA5-22294, PA5-18147 and MAB-62191 for the binding to TRIM21, in particular for the binding to contiguous amino acid residues localized within the coiled-coil domain of TRIM21, in particular the coiled-coil domain localized between amino acid residues 128 and 238 of TRIM21 of SEQ ID No. 2 or the coiled-coil domain of amino acid of SEQ ID No. 4, and/or for the binding to contiguous amino acid residues localized within the PRY-SPRY domain of TRIM21, in particular the PRYSPRY domain localized between amino acid residues 268 and 465 of TRIM21 of SEQ ID No. 2 or the PRY-SPRY domain corresponding to amino acid sequence of SEQ ID No. 5, the antibody, antigen-binding fragment thereof or antigen-binding antibody mimetic enhancing the phagocytosis of cells, particularly TRIM21-positive cells, particularly tumor cells and/or secondary necrotic cells by myeloid cells, in particular by dendritic cells and/or macrophages, as compared to the competing antibody.

To determine if a test antibody can compete for binding to the same target bound by the at least one competing antibody selected among the list consisting of anti-TRIM21 antibodies sc-25351, PA5-22294, PA5-18147 and MAB-62191, a cross-blocking assay (e.g. a competitive ELISA assay) can be performed. In an exemplary competitive ELISA assay, a polypeptide comprising or consisting of the coiled-coil domain localized between amino acid residues 128 and 238 of TRIM21 of SEQ ID No. 2 or the coiled-coil domain corresponding to amino acid residues of SEQ ID No. 4, and/or which binds to an epitope sequence localized within the PRY-SPRY domain of TRIM21, in particular the PRY-SPRY domain localized between amino acid residues 268 and 465 of TRIM21 of SEQ ID No. 2 or the PRY-SPRY domain corresponding to amino acid residues of SEQ ID No. 5; may be coated on the wells of a microtiter plate and pre-incubated with or without candidate competing antibody and then a biotin-labeled candidate antibody is added. The amount of labeled candidate antibody bound to the polypeptide is measured using avidin-peroxidase conjugate and appropriate substrate. The antibody can be labeled with a radioactive or fluorescent label or some other detectable and measurable label. The amount of labeled candidate antibody that bound to the polypeptide will have an indirect correlation to the ability of the candidate antibody to compete for binding to the same epitope, i.e., the greater the affinity of the candidate antibody for the target, the less labeled competing antibody will be bound to the polypeptide-coated wells. A candidate antibody is considered an antibody that competes for binding to the same polypeptide as competing antibody of the invention when the candidate antibody can block binding of the competing antibody by at least 20%, preferably by at least 20-50%, even more preferably, by at least 50% as compared to a control performed in parallel in the absence of the candidate antibody (but may be in the presence of a known non-competing antibody). It will be understood that variations of this assay can be performed to arrive at the same quantitative value.

Of course, depending on the competing antibody, the polypeptide coated on the plate may vary to correspond more closely to the domain recognized by the competing antibodies.

In a particular embodiment of the invention, the compound is an antibody, an antigen-binding fragment thereof or an antigen-binding antibody mimetic, which binds to TRIM21 especially binds specifically to TRIM21, and which antagonizes the binding between CLEC1 and TRIM21, in particular between human CLEC-1 and human TRIM21, said antibody, antigen-binding fragment thereof or antigen-binding antibody mimetic competes with the competing antibody PA5-22294 for the binding to the amino acid sequence set forth in SEQ ID No. 6, the antibody, antigen-binding fragment thereof or antigen-binding antibody mimetic enhancing the phagocytosis of cells, particularly TRIM21-positive cells, particularly tumor cells and/or secondary necrotic cells by myeloid cells, particularly TRIM21-positive tumor cells and/or secondary necrotic cells, by myeloid cells in particular by dendritic cells and/or macrophages, as compared to the competing antibody.

In a particular embodiment of the invention, the compound is an antibody, an antigen-binding fragment thereof or an antigen-binding antibody mimetic, which binds to TRIM21 especially binds specifically to TRIM21, and which antagonizes the binding between CLEC1 and TRIM21, in particular between human CLEC-1 and human TRIM21, said antibody, antigen-binding fragment thereof or antigen-binding antibody mimetic competes with the competing antibody sc-25351 for the binding to the amino acid sequence set forth in SEQ ID No. 7, the antibody, antigen-binding fragment thereof or antigen-binding antibody mimetic enhancing the phagocytosis of cells, particularly TRIM21-positive cells, particularly tumor cells and/or secondary necrotic cells by myeloid cells, particularly TRIM21-positive tumor cells and/or secondary necrotic cells, in particular by dendritic cells and/or macrophages, as compared to the competing antibody.

In a particular embodiment of the invention, the compound is an antibody, an antigen-binding fragment thereof or an antigen-binding antibody mimetic, which binds to TRIM21, especially binds specifically to TRIM21, and which antagonizes the binding between CLEC1 and TRIM21, in particular between human CLEC-1 and human TRIM21, said antibody, antigen-binding fragment thereof or antigen-binding antibody mimetic competes with the competing antibody MAB6219 for the binding to the amino acid sequence set forth in SEQ ID No. 8, the antibody, antigen-binding fragment thereof or antigen-binding antibody mimetic enhancing the phagocytosis of cells, particularly TRIM21-positive cells, particularly tumor cells and/or secondary necrotic cells by myeloid cells, particularly TRIM21-positive tumor cells and/or secondary necrotic cells; in particular by dendritic cells and/or macrophages, as compared to the competing antibody.

In a particular embodiment of the invention, the compound is an antibody, an antigen-binding fragment thereof or an antigen-binding antibody mimetic, which binds to TRIM21, especially binds specifically to TRIM21, and which antagonizes the binding between CLEC1 and TRIM21, in particular between human CLEC-1 and human TRIM21, said antibody, antigen-binding fragment thereof or antigen-binding antibody mimetic competes with the competing antibody PA5-18147 for the binding to the amino acid sequence set forth in SEQ ID No. 9, the antibody, antigen-binding fragment thereof or antigen-binding antibody mimetic enhancing the phagocytosis of cells, particularly TRIM21-positive cells, particularly tumor cells and/or secondary necrotic cells by myeloid cells, particularly TRIM21-positive tumor cells and/or secondary necrotic cells, in particular by dendritic cells and/or macrophages, as compared to the competing antibody.

The expression “small organic compounds” encompasses a molecule of a size comparable to those organic molecules generally used in pharmaceuticals. The term excludes biological macromolecules (e.g., proteins, nucleic acids, etc.). Preferred small organic molecules range in size up to about 5000 Da, more in particular up to 2000 Da, and most in particular up to about 1000 Da.

As used herein, the term “antibody” comprises polyclonal antibodies, monoclonal antibodies or recombinant antibodies. As used herein, a “monoclonal antibody” is intended to refer to a preparation of antibody molecules to obtain antibodies which share a common heavy chain and common light chain amino acid sequence, in contrast with “polyclonal” antibody preparations which contain a mixture of antibodies of different amino acid sequence. Monoclonal antibodies can be generated by several known technologies like phage, bacteria, yeast or ribosomal display, as well as by classical methods exemplified by hybridoma-derived antibodies. They may also be synthetized using the disclosed amino acid sequences as reference. Thus, the term “monoclonal” is used to refer to all antibodies derived from one nucleic acid clone.

As used herein, the term “antibody” further designates antibodies which have been modified as compared to a wild type antibody; and encompasses chimeric antibodies, humanized antibodies, fully humanized antibodies, de-immunized antibodies, modified antibodies, and antigen-binding antibody mimetics. In a particular embodiment of the invention, the compound is an antibody or an antigen-binding fragment thereof, which binds to TRIM21, especially binds specifically to TRIM21, and which comprises a human IgG1, IgG2, IgG3 or IgG4 constant region.

The antibodies of the present invention include recombinant antibodies. As used herein, the term “recombinant antibody” refers to antibodies which are produced, expressed, generated or isolated by recombinant means, such as antibodies which are expressed using a recombinant expression vector transfected into a host cell; antibodies isolated from a recombinant combinatorial antibody library; antibodies isolated from an animal (e.g. a mouse) which is transgenic due to human immunoglobulin genes; or antibodies which are produced, expressed, generated or isolated in any other way in which particular immunoglobulin gene sequences (such as human immunoglobulin gene sequences) are assembled with other DNA sequences. Recombinant antibodies include, for example, chimeric and humanized antibodies.

As used herein, a “chimeric antibody” refers to an antibody in which the sequence of the variable domain derived from the germline of a mammalian species, such as a mouse, have been grafted onto the sequence of the constant domain derived from the germline of another mammalian species, such as a human.

As used herein, a “humanized antibody” refers in a first embodiment to an antibody in which CDR sequences derived from the germline of another mammalian species, such as a mouse, have been grafted onto human or especially humanized framework sequences. In a further embodiment, a “humanized antibody” refers to an antibody wherein at least one CDR and all or part of framework sequences have been humanized.

As used herein, an “antigen-binding fragment of an antibody” means a part of an antibody, i.e. a molecule corresponding to a portion of the structure of the antibody of the invention, that exhibits antigen-binding capability for TRIM21 or CLEC-1, possibly in its native form; such fragment especially exhibits the same or substantially the same antigen-binding specificity for said antigen compared to the antigen-binding specificity of the corresponding four-chain antibody. Advantageously, the antigen-binding fragments have a similar binding affinity as the corresponding 4-chain antibodies. However, antigen-binding fragment that have a reduced antigen-binding affinity with respect to corresponding 4-chain antibodies are also encompassed within the invention. The antigen-binding capability can be determined by measuring the affinity between the antibody and the target fragment. These antigen-binding fragments may also be designated as “functional fragments” of antibodies.

Antigen-binding fragments of antibodies are fragments which comprise their hypervariable domains designated CDRs (Complementary Determining Regions) or part(s) thereof encompassing the recognition site for the antigen, i.e. TRIM21. Antigen binding fragments of an antibody that contain the variable domains comprising the CDRs of said antibody encompass Fv, dsFv, scFv, Fab, Fab′, F(ab′)2. These basic antigen-binding fragments of the invention can be combined together to obtain multivalent antigen-binding fragments, such as diabodies, tribodies or tetrabodies. These multivalent antigen-binding fragments are also part of the present invention.

Antigen-binding antibody mimetics are organic compounds that specifically bind antigens, but that are not structurally related to antibodies. They are usually artificial peptides or small proteins with a molar mass of about 3 to 20 kDa. Nucleic acids and small molecules are sometimes considered antibody mimetics as well, but not artificial antibodies, antibody fragments and fusion proteins composed from these. Common advantages over antibodies are better solubility, tissue penetration, stability towards heat and enzymes, and comparatively low production costs. Antibody mimetics are being developed as therapeutic and diagnostic agents. Antigen-binding antibody mimetics may also be selected among the group comprising affibodies, affilins, aptamers, anticalins, affimers, affitins, DARPins, and Monobodies.

As used herein, the term “polypeptide” means a polymer of amino acids having every or any length in amino acid residues. Thus, peptides, oligopeptides and proteins are included in the definition of “polypeptide” and these terms are used interchangeably throughout the specification, as well as in the claims. The term “polypeptide” does not exclude post-translational modifications that include but are not limited to phosphorylation, acetylation, glycosylation and the like.

The term “a functionally equivalent fragment” as used herein may mean any fragment or assembly of fragments of TRIM21, or of CLEC-1 that binds to TRIM21, especially binds specifically to TRIM21. Accordingly, the present invention provides a polypeptide, in particular a functional equivalent, capable of lowering/reducing or inhibiting binding of CLEC-1 to TRIM21, which polypeptide comprises consecutive amino acids having a sequence which is the sequence of at least a portion of a domain of TRIM21 that binds to CLEC-1 for a functional equivalent of TRIM21 or of at least a portion of an extracellular domain of CLEC-1 for a functional equivalent of CLEC-1, which portion binds to TRIM21 especially binds specifically to TRIM21. A functional equivalent of CLEC-1 has been defined above and encompasses in particular a fragment of the extracellular domain of CLEC1 of amino acid sequence of SEQ ID No. 3 or a variant thereof which is a polypeptide having at least or more than 80%, in particular at least or more than 85% in particular at least or more than 90% identity with the aligned amino acid sequence in the sequence of SEQ ID No. 3.

In some embodiments, the functional equivalent is fused to a heterologous polypeptide to form a fusion protein. As used herein, a “fusion protein” comprises all or part (typically biologically active) of a functional equivalent of the present invention operably linked to a heterologous polypeptide (i.e., a polypeptide other than the same polypeptide). Within the fusion protein, the term “operably linked” is intended to indicate that the functional equivalent of the present invention and the heterologous polypeptide are fused in-frame to each other. The heterologous polypeptide can be fused to the N-terminus or C-terminus of the functional equivalent of the present invention.

In some embodiments, the functional equivalent is fused to an immunoglobulin constant domain (Fc region) to form an immunoadhesin. Immunoadhesins can possess many of the valuable chemical and biological properties of human antibodies. Since immunoadhesins can be constructed from a human protein sequence with a desired specificity linked to an appropriate human immunoglobulin hinge and constant domain (Fc) sequence, the binding specificity of interest can be achieved using entirely human components. Such immunoadhesins are minimally immunogenic to the patient, and are safe for chronic or repeated use. In some embodiments, the Fc region is a native sequence Fc region. In some embodiments, the Fc region is a variant Fc region. In still another embodiment, the Fc region is a functional Fc region. As used herein, the term “Fc region” is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The adhesion portion and the immunoglobulin sequence portion of the immunoadhesin may be linked by a minimal linker. The immunoglobulin sequence typically, but not necessarily, is an immunoglobulin constant domain. The immunoglobulin moiety in the chimeras of the present invention may be obtained from IgG1, IgG2, IgG3 or IgG4 subtypes, IgA, IgE, IgD or IgM, but typically IgG1 or IgG4. In some embodiments, the functional equivalent of CLEC-1 or TRIM21 and the immunoglobulin sequence portion of the immunoadhesin are linked by a minimal linker. As used herein, the term “linker” refers to a sequence of at least one amino acid that links the polypeptide of the invention and the immunoglobulin sequence portion. Such a linker may be useful to prevent steric hindrances. In some embodiments, the linker has 4; 5; 6; 7; 8; 9; 10; 11; 12; 13; 14; 15; 16; 17; 18; 19; 20; 21; 22; 23; 24; 25; 26; 27; 28; 29; 30 amino acid residues. However, the upper limit is not critical but is chosen for reasons of convenience regarding e.g. biopharmaceutical production of such polypeptides. The linker sequence may be a naturally occurring sequence or a non-naturally occurring sequence. If used for therapeutic purposes, the linker is typically non-immunogenic in the subject to which the immunoadhesin is administered. One useful group of linker sequences are linkers derived from the hinge region of heavy chain antibodies as described in WO 96/34103 and WO 94/04678. Other examples are poly-alanine linker sequences.

In a particular embodiment of the invention, the antagonist compound is a functional equivalent of TRIM21. Functional equivalents of TRIM21 include but are not limited to molecules that bind to CLEC-1 and comprise all or a portion TRIM21 so as to form a soluble receptor that is capable to bind to CLEC-1. Such a functional equivalent of TRIM21 may be a peptide or a polypeptide. In particular, such a functional equivalent of TRIM21 may be a peptide or a polypeptide that comprises at least 20, in particular at least 25, in particular at least 30, in particular at least 40, in particular 50 amino acid residues, in particular at least 80 amino acid residues, in particular at least 100 amino acid residues, in particular at least 200 amino acid residues, in particular at least 300 contiguous amino acid residues within sequence SEQ ID No.2. Alternatively, the antagonist is a peptide or a polypeptide that comprises at least 20 amino acid residues, in particular at least 25 amino acid residues, in particular at least 30 amino acid residues, in particular at least 40 amino acid residues, in particular 50 amino acid residues, in particular at least 80 amino acid residues, in particular at least 100 amino acid residues, in particular at least 200 amino acid residues, in particular at least 300 and has at least 80% identity, more particularly at least 85%, at least 86%, at least 87%, at least 88%, at least 89%, at least 90%, at least 91%, at least 92%, at least 93%, at least 94%, at least 95%, at least 96%, at least 97%, at least 98% and even more particularly at least 99% of identity, or share 100% identity, with the TRIM21 or a corresponding protein, in particular TRIM21 of SEQ ID No. 2. The antagonist compound may be a polypeptide fused to an immunoglobulin constant domain, which comprises of consists of an amino acid sequence having at least 80% identity, more particularly at least 85%, at least 86%, at least 87%, at least 88%, at least 89%, at least 90%, at least 91%, at least 92%, at least 93%, at least 94%, at least 95%, at least 96%, at least 97%, at least 98% and even more particularly at least 99% or share 100% identity, with TRIM21 or a corresponding protein, in particular with TRIM21 of SEQ ID No. 2. As used herein, the term “corresponding protein” refers to the protein for which as defined herein the functional equivalent of the invention has similar function. The percentages of identity to which reference is made in the presentation of the present invention are determined on the basis of a global alignment of sequences to be compared, that is to say, on an alignment of sequences over their entire length, using for example the algorithm of Needleman and Wunsch 1970. This sequence comparison can be done for example using the needle software by using the parameter “Gap open” equal to 10.0, the parameter “Gap Extend” equal to 0.5, and a matrix “BLOSUM 62”. Software such as needle is available on the website ebi.ac.uk worldwide, under the name “needle”.

Functional equivalents of CLEC-1 include but are not limited to molecules that bind to TRIM21 and comprise all or a portion of the extracellular domain of CLEC-1 so as to form a soluble receptor that is capable to bind to TRIM21. Thus, the functional equivalents include soluble forms of CLEC-1. A suitable soluble form of these proteins, or functional equivalents thereof, may comprise, for example, a truncated form of the protein from which the transmembrane domain has been removed by chemical, proteolytic or recombinant methods and may comprise or consist in the extracellular domain of CLEC-1 or a fragment thereof as defined herein. Particularly, the functional equivalent consists of an amino acid sequence having at least or more than 80% identity, more particularly at least or more than 85%, at least or more than 86%, at least or more than 87%, at least or more than 88%, at least or more than 89%, at least or more than 90%, at least or more than 91%, at least or more than 92%, at least or more than 93%, at least or more than 94%, at least or more than 95%, at least or more than 96%, at least or more than 97%, at least or more than 98% and even more particularly at least 99% of identity with the corresponding protein over the entire length of the corresponding protein, in particular having at least or more than 80% or at least or more than 85% and especially at least or more than 90% identity with the aligned amino acid sequence No.3. As used herein, the term “corresponding protein” refers to the protein for which the functional equivalent of the invention has similar function. The percentages of identity to which reference is made in the presentation of the present invention are determined on the basis of a global alignment of sequences to be compared, that is to say, on an alignment of sequences over their entire length, using for example the algorithm of Needleman and Wunsch 1970. This sequence comparison can be done for example using the needle software by using the parameter “Gap open” equal to 10.0, the parameter “Gap Extend” equal to 0.5, and a matrix “BLOSUM 62”. Software such as needle is available on the website ebi.ac.uk worldwide, under the name “needle”.

The antagonist compound is provided for use as a medicament. In particular, the antagonist compound is provided for use in the treatment of a disease in a subject (i.e. a patient, in particular a human patient), in particular a subject having a cancer, a sepsis or an infectious disease. In another embodiment, the antagonist compound may be provided for use in the prevention of a disease in a subject. As used herein, “treatment” or “treating” is an approach for obtaining beneficial or desired results. Beneficial or desired clinical results include, but are not limited to, one or more of the following: alleviating, preventing or abolishing one or more symptoms resulting from the disease, curing the disease, diminishing the extent of the disease, stabilizing the disease (e.g., preventing or delaying the worsening of the disease), preventing or delaying the spread of the disease, preventing or delaying the recurrence of the disease, delaying or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. According to an embodiment, the term “treatment” relates to the prophylactic treatment. As used herein, the term “prevent” refers to the reduction in the risk of acquiring or developing a given condition.

In a particular embodiment of the invention, the antagonist compound is provided for use in the treatment of a patient, wherein the modulation of the phagocytosis of target cells, in particular such as cells expressing TRIM21, more particularly of TRIM21-positive tumor cells, by myeloid cells, in particular by dendritic cells and/or macrophages, to improve the outcome of a disease. Thus, in a particular embodiment, it is provided an antagonist compound to increase the phagocytosis of cells, in particular of TRIM21-positive cells, more particularly of tumor cells and/or secondary necrotic cells, even more particularly of TRIM21-positive tumor cells and/or TRIM21-positive secondary necrotic cells, by myeloid cells, particularly by dendritic cells and/or macrophages. Thus, TRIM21-positive tumors (i.e. tumors that include at least one cell expressing, in particular abnormally expressing, TRIM21) can be targeted by the compound of the invention. Indeed, it has been shown that antagonists of TRIM21-CLEC1 signaling pathway (e.g. compounds binding to TRIM21 or CLEC1 that inhibits the binding between CLEC-1 and TRIM21, or functional equivalent of TRIM21 or CLEC1, or compounds that reduce the expression of functional TRIM21 or of CLEC1, or compounds that reduce or inhibit the signaling pathway induced by the binding between TRIM21 and CLEC-1) are able to modulate, in particular to enhance, the phagocytosis of target cells, such as tumor cells expressing TRIM21 by myeloid cells, in particular by dendritic cells and/or macrophages, thereby leading to their use in therapy or treating disease wherein modulating the phagocytosis of target cells such as tumor cells or cells expressing TRIM21 by myeloid cells, in particular dendritic cells and/or macrophages, improves the health of the patient. Thereby, the use of an antagonist compound of TRIM21, which at least reduces the interaction of CLEC-1 (expressed on the cell surface of dendritic cells and macrophages) to its ligand TRIM21 (expressed for example at least by tumor cells), could be useful for modulating the phagocytosis of cells such as tumor cells by myeloid cells, in particular dendritic cells and/or macrophages. When CLEC-1A-expressing myeloid cells, in particular macrophages or dendritic cells, interact with cells expressing TRIM21, the phagocytosis by these macrophages or dendritic cells may be inhibited or reduced. As an example, it has been shown that Tumor cells (i.e. Raji cells) that express TRIM21 escape phagocytosis exerted by macrophages. By using an antagonist compound according to the invention that modulates the interaction between CLEC-1-expressing myeloid cells and TRIM21-expressing cells, in particular TRIM21-positive tumor cells, the reduction or inhibition of the phagocytosis of TRIM21-expressing cells by myeloid cells, in particular dendritic cells or macrophages, is lowered. Consequently, the phagocytosis of TRIM21-expressing cells, like tumor cells, is enhanced in presence of an antagonist compound of the invention.

More particularly, the present invention concerns the use of the antagonist compound, in the treatment of a condition or a disease in a patient wherein said condition or disease is, or is related to, a cancer listed in the present description, in particular cancer with TRIM21-positive tumor cells, such as breast cancer, hepatocellular carcinoma, lymphoma, more particularly B-cell lymphoma, colon cancer, thyroid cancer, liver cancer, renal cancer, melanoma, colorectal cancer, nasopharyngeal carcinoma, pancreatic cancer, a chronic infection, a sepsis, an infection, in particular by a Coxsackievirus or by an encephalitis virus, more particularly by Coxsackievirus B3 or Japanese encephalitis virus, a cardiovascular disease, an auto-immune disease, in particular Sjogren's syndrome or systemic lupus erythematosus or systemic sclerosis, an inflammatory disease.

The term “cancer” has its general meaning in the art and refers to a group of diseases involving abnormal cell growth with the potential to invade or spread to other parts of the body. The term “cancer” further encompasses both primary and metastatic cancers. Examples of cancers that may treated by methods and compositions of the invention include, but are not limited to, cancer with TRIM21-positive tumor cells, cancer with TRIM21-positive tumor, cancer from the bladder, blood, bone, bone marrow, brain, breast, colon, oesophagus, gastrointestinal, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; non encapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malign melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brennertumor, malignant; phyllodestumor, malignant; synovial sarcoma; mesothelioma, malignant; dysgerminoma; embryonal carcinoma; teratoma, malignant; strumaovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblasticodontosarcoma; ameloblastoma, malignant; ameloblasticfibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukaemia; lymphoid leukaemia; plasma cell leukaemia; erythroleukemia; lymphosarcoma cell leukaemia; myeloid leukaemia; basophilic leukaemia; eosinophilic leukaemia; monocyticleukaemia; mast cell leukaemia; megakaryoblasticleukaemia; myeloid sarcoma; and hairy cell leukaemia.

In a particular embodiment, the subject suffers from a cancer selected from the group consisting of bile duct cancer, bladder cancer, bone cancer, brain and central nervous system cancer, breast cancer, Castleman disease cervical cancer, colorectal cancer, endometrial cancer, oesophagus cancer, gallbladder cancer, gastrointestinal carcinoid tumors, Hodgkin's disease, non-Hodgkin's lymphoma, Kaposi's sarcoma, kidney cancer, laryngeal and hypopharyngeal cancer, liver cancer, lung cancer, mesothelioma, plasmacytoma, nasal cavity and paranasal sinus cancer, nasopharyngeal cancer, neuroblastoma, oral cavity and oropharyngeal cancer, ovarian cancer, pancreatic cancer, penile cancer, pituitary cancer, prostate cancer, retinoblastoma, rhabdomyosarcoma, salivary gland cancer, skin cancer, stomach cancer, testicular cancer, thymus cancer, thyroid cancer, vaginal cancer, vulvar cancer, and uterine cancer.

The present invention also concerns the use of the compound in the treatment, including the preventive treatment, of a deleterious condition or a disease, in particular wherein the dendritic cells and/or the T cells are involved, and wherein the proliferation of T cells and/or the stimulation of the phagocytosis by myeloid cells, in particular by dendritic cells and/or macrophages, may improve or treat the condition or the disease.

In a particular embodiment, the disease or condition is selected from the group consisting of cancer, in particular a cancer as listed here above, more particularly cancers with TRIM21-positive tumor cells, liquid cancers, solid cancers, lymphoma, colorectal cancers, mesothelioma or hepatocarcinoma.

In a particular embodiment of the invention a compound is provided that is an antagonist of the binding between CLEC-1 and TRIM21, in particular an antagonist of the binding between human CLEC-1 and human TRIM21, for use in the treatment of a human subject suffering from a cancer with TRIM21 positive tumor cells.

In other words, in a particular embodiment the invention relates to a compound that is an antagonist of the binding between CLEC-1 and TRIM21, in particular an antagonist of the binding between human CLEC-1 and human TRIM21, for use in the treatment of a cancer with TRIM21 positive tumor cells, in particular in a human subject suffering from such cancer with TRIM21 positive tumor cells.

In a particular embodiment, such compound for use in the treatment of a human subject suffering from a cancer with TRIM-1 positive tumor cells or for use in the treatment of a cancer with TRIM21 positive tumor cells, binds to CLEC-1, in particular to human CLEC-1, especially specifically binds to CLEC-1, in particular specifically binds to human CLEC-1, or is a functional equivalent of CLEC-1 in particular of human CLEC-1 as defined above in particular is a polypeptide.

In a particular embodiment, the invention relates to such antagonist compound, which is an antagonist of the binding between CLEC-1 and TRIM21, for use in the treatment of a human subject suffering from a cancer with TRIM-1 positive tumor cells or for use in the treatment of a cancer with TRIM21 positive tumor cells, by increasing the phagocytosis of TRIM 21-positive tumor cells, in particular by dendritic cells and/or macrophages.

In a particular embodiment the compound for use in the treatment of a cancer with TRIM21-positive tumor cells, in a patient suffering from cancer, is in particular for use in the treatment of cancer selected from breast cancer, hepatocellular carcinoma, lymphoma, more particularly B-cell lymphoma, colon cancer, thyroid cancer, liver cancer, renal cancer, melanoma, colorectal cancer, nasopharyngeal carcinoma, pancreatic cancer.

The invention also relates to the use of a compound that is an antagonist of the binding between CLEC-1 and TRIM21, in particular an antagonist of the binding between human CLEC-1 and human TRIM21, for the manufacture of a medicament for the treatment of a human subject suffering from a cancer with TRIM21 positive tumor cells.

The invention also relates to the use of a compound that is an antagonist of the binding between CLEC-1 and TRIM21, in particular an antagonist of the binding between human CLEC-1 and human TRIM21, for the manufacture of a medicament for the treatment of a cancer with TRIM21 positive tumor cells, in particular in a human subject suffering from such cancer with TRIM21 positive tumor cells. The compound is as defined in any of the embodiments disclosed herein.

The invention also relates to a method of treating cancer with TRIM21 positive tumor cells, in a human subject in need thereof, comprising the administration to said subject of an effective amount of a compound that is an antagonist of the binding between CLEC-1 and TRIM21, in particular an antagonist of the binding between human CLEC-1 and human TRIM21. The compound is as defined in any of the embodiments disclosed herein.

The invention also relates to a method of treating a human subject suffering from a cancer with TRIM21 positive tumor cells, comprising the administration to said subject of an effective amount of a compound that is an antagonist of the binding between CLEC-1 and TRIM21, in particular an antagonist of the binding between human CLEC-1 and human TRIM21. The compound is as defined in any of the embodiments disclosed herein.

The invention also relates to a method for increasing the phagocytosis of TRIM21-positive cells, more particularly of TRIM21-positive tumor cells and/or secondary necrotic cells, especially by dendritic cells and/or macrophages, comprising the administration in a patient in need thereof of an effective amount of a compound of the invention, in particular wherein said compound is an antagonist of the binding between CLEC-1 and TRIM21. The compound is as defined in any of the embodiments disclosed herein.

In another particular embodiment of the invention, it is provided a compound that inhibits the C-type lectin-like receptor-1 (CLEC-1)/tripartite motif-containing protein 21 (TRIM21) signaling pathway, wherein the compound binds to CLEC1, in particular specifically binds to CLEC-1, or reduces the expression of functional CLEC1 or is a functional equivalent of CLEC1, especially a polypeptide which is a functional equivalent of CLEC-1, in particular the compound is an antagonist compound of the binding between CLEC-1 and TRIM21 that binds to CLEC1, more particularly binds to human CLEC1, especially specifically binds to human CLEC-1, and in a more particular embodiment binds specifically to the extracellular domain of human CLEC-1, for use in the treatment of a subject, in particular a human subject, suffering from a cancer with TRIM21-positive tumor cells. Preferably said compound increases the phagocytosis capability of myeloid cells, in particular dendritic cells and/or macrophages, in particular which increases the phagocytosis of TRIM21-positive cells, more particularly of TRIM21-positive tumor cells and/or secondary necrotic cells, by dendritic cells and/or macrophages, in particular which increases the phagocytosis of tumor cells and/or secondary necrotic cells by dendritic cells and/or macrophages. In particular said compound is selected from an antibody, an antigen-binding fragment thereof, an antigen-binding antibody mimetic, which binds to CLEC-1, in particular to human CLEC-1, and accordingly especially specifically binds to CLEC-1 said compound being an antagonist of the binding between CLEC-1 and TRIM21, in particular between human CLEC-1 and human TRIM21, which competes for the binding to CLEC-1, in particular to human CLEC-1, with an anti-CLEC-1A antibody having the heavy chain variable domain of SEQ ID No. 10 and the light chain variable domain of SEQ ID No. 11, compound enhancing the phagocytosis capability of myeloid cells, in particular dendritic cells and/or macrophages, in particular the phagocytosis of TRIM21-positive cells, more particularly of TRIM21-positive tumor cells and/or secondary necrotic cells, by dendritic cells and/or macrophages, more particularly the phagocytosis of tumor cells and/or secondary necrotic cells by dendritic cells and/or macrophages.

In a particular embodiment of the invention, it is provided a compound selected from an antibody, an antigen-binding fragment thereof, an antigen-binding antibody mimetic, which binds to CLEC-1, in particular to human CLEC-1, and accordingly especially specifically binds to CLEC-1 said compound being an antagonist of the binding between CLEC-1 and TRIM21, in particular between human CLEC-1 and human TRIM21, which competes for the binding to CLEC-1, in particular to human CLEC-1, with an anti-CLEC-1 antibody having the heavy chain variable domain of SEQ ID No. and the light chain variable domain of SEQ ID No. 11, in particular said compound enhancing the phagocytosis capability of myeloid cells, in particular dendritic cells and/or macrophages, in particular the phagocytosis of TRIM21-positive cells, more particularly of TRIM21-positive tumor cells and/or secondary necrotic cells, by dendritic cells and/or macrophages, more particularly the phagocytosis of tumor cells and/or secondary necrotic cells by dendritic cells and/or macrophages,

for use in the treatment of a patient suffering from a cancer, in particular cancer with TRIM21-positive tumor cells. Cancers that may be treated encompass as examples breast cancer, hepatocellular carcinoma, lymphoma, more particularly B-cell lymphoma, colon cancer, thyroid cancer, liver cancer, renal cancer, melanoma, colorectal cancer, nasopharyngeal carcinoma, pancreatic cancer.

In a particular embodiment of the invention, it is provided a compound selected from an antibody, an antigen-binding fragment thereof, an antigen-binding antibody mimetic, which binds to CLEC-1, in particular to human CLEC-1, and accordingly especially specifically binds to CLEC-1 (in particular to its extracellular domain) said compound being an antagonist of the binding between CLEC-1 and TRIM21, in particular between human CLEC-1 and human TRIM21, which competes for the binding to CLEC-1, in particular to human CLEC-1, with an anti-CLEC-1 antibody having the heavy chain variable domain of SEQ ID No. 10 and the light chain variable domain of SEQ ID No. 11, in particular said compound enhancing the phagocytosis capability of myeloid cells, in particular dendritic cells and/or macrophages, in particular the phagocytosis of TRIM21-positive cells, more particularly of TRIM21-positive tumor cells and/or secondary necrotic cells, by dendritic cells and/or macrophages, more particularly the phagocytosis of tumor cells and/or secondary necrotic cells by dendritic cells and/or macrophages, for use in the treatment of a patient having a cancer with TRIM21-positive tumor cells.

The antibody referenced anti-CLEC-1 mAb #5 (also designated αCLEC-1 #5) in the working examples of the invention has the heavy chain variable domain of SEQ ID No. and the light chain variable domain of SEQ ID No. 11. Competition for the binding to CLEC-1 may be assessed by a cross-blocking assay (e.g. a competitive ELISA assay). In an exemplary competitive ELISA assay, a polypeptide comprising or consisting of at least the extracellular domain of CLEC-1; may be coated on the wells of a microtiter plate and pre-incubated with or without candidate competing antibody and then a biotin-labeled candidate antibody is added. The amount of labeled candidate antibody bound to the polypeptide is measured using avidin-peroxidase conjugate and appropriate substrate. The antibody can be labeled with a radioactive or fluorescent label or some other detectable and measurable label. The amount of labeled candidate antibody that bound to the polypeptide will have an indirect correlation to the ability of the candidate antibody to compete for binding to the same epitope, i.e., the greater the affinity of the candidate antibody for the target, the less labeled competing antibody will be bound to the polypeptide-coated wells. A candidate antibody is considered an antibody that competes for binding to the same polypeptide as competing antibody of the invention when the candidate antibody can block binding of the competing antibody by at least 20%, preferably by at least 20-50%, even more preferably, by at least 50% as compared to a control performed in parallel in the absence of the candidate antibody (but may be in the presence of a known non-competing antibody). It will be understood that variations of this assay can be performed to arrive at the same quantitative value.

When the compound that binds CLEC-1, in particular human CLEC-1, especially specifically binds human CLEC-1, is an antibody, it may be a chimeric antibody, a humanized antibody, a recombinant antibody, a fully-humanized antibody, a monoclonal antibody, a de-immunized antibody, in particular a humanized monoclonal antibody.

In a particular embodiment, the invention relates to a compound that is a polypeptide that is a functional equivalent of CLEC-1, in particular of human CLEC-1, as disclosed herein, in particular such a polypeptide fused to an immunoglobulin constant domain.

### Pharmaceutical Compositions

The invention also concerns a pharmaceutical composition for use in the treatment of a patient having a disease, comprising as a therapeutic agent an antagonist of CLEC-1/TRIM21 signaling pathway, in particular an antagonist compound which binds to TRIM21, especially binds specifically to TRIM21, as defined herein, or a CLEC-1A antagonist which is an antibody or antigen-binding fragment or an antigen-binding antibody mimetic thereof which binds to CLEC-1 and accordingly especially specifically binds to CLEC-1 according to any embodiment disclosed herein, either alone or in combination with a second therapeutic agent, with a pharmaceutical suitable vehicle, which are pharmaceutically acceptable for a formulation capable of being administered to a patient in need thereof. These formulations may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

### Combinations of Compounds

The invention also concerns a combination of compounds comprising a first therapeutic agent and at least one further therapeutic agent.

A combination of compounds is a formulation comprising at least two different products, or agents, or compounds, which may be packed together or packed separately, and that are prepared for a simultaneous administration, a co-administration, or a coordinated administration (or sequential administration). In particular, the amount of each compound within the combination may be comprised between 1 μg/kg and 100 mg/kg of weight of the patient.

The first therapeutic agent is an antagonist as defined in any embodiment of the invention disclosed herein (i.e. an antagonist that binds to TRIM21, especially binds specifically to TRIM21, like antibody, antigen-binding fragment thereof or antigen-binding antibody mimetic as disclosed in an embodiment of the invention, a compound that reduce or inhibit the signaling pathway activated following the interaction between TRIM21 and CLEC-1, a compound that reduces the expression of TRIM21, etc.).

At least one further therapeutic agent is selected from the list consisting of a tumor-targeting antibody or antigen-binding fragment thereof, in particular a tumor-targeting monoclonal antibody or antigen-binding fragment thereof, more particularly a tumor-targeting monoclonal antibody or antigen-binding fragment thereof which activates and/or enhances the phagocytosis of tumor cells or cells expressing TRIM21 by myeloid cells, in particular by dendritic cells and/or macrophages, and still more particularly a monoclonal antibody selected from the group consisting of alemtuzumab, atezolizumab, bevacizumab, cetuximab, herceptin, panitumumab, rituximab, trastuzumab, an anti-PDL-1 antibody, and an anti-CD47 antibody, or another antibody or monoclonal antibody selected from the group consisting of an anti-PD1 antibody and an anti-SIRPa antibody; and/or a chemotherapeutic agent, in particular a cytotoxic agent with anti-proliferative, pro-apoptotic, cell cycle arresting and/or differentiation inducing effect, more particularly a cytotoxic agent selected from the group consisting of cytotoxic antibody, alkylating drugs, anthracyclines, antimetabolites, anti-microtubule agents, topoisomerase inhibitors, alkaloids, bleomycin, antineoplastic drugs, cyclophosphamide. A tumor-targeting antibody may be defined as a therapeutic monoclonal antibody that recognizes tumor-specific membrane proteins, blocks cell signalling, and induces tumor killing through Fc-driven innate immune responses. The chemotherapeutic agent may be a conventional cytotoxic agent, i.e. a compound that induces irreversible lethal lesions through interference with DNA replication, mitosis, etc. following exposure. These agents may have anti-proliferative, pro-apoptotic, cell cycle arresting, and differentiation inducing effects. These agents are preferentially selected from the group consisting of alkylating drugs (cisplatin, chlorambucil, procarbazine, carmustine), anthracyclines and other cytotoxic antibiotics, antimetabolites (i.e. methotrexate, cytarabine, gemcitabine), anti-microtubule agents (i.e. vinblastine, paclitaxel, docetaxel), topoisomerase inhibitors (i.e. etoposide, doxorubicin), alkaloids (i.e. Vincristine, Vinblastine, Vinorelbine, Camptothecin) or bleomycin (inhibiting incorporation of thymidine into DNA strands). The combination may comprise more than one second therapeutic agent selected from the list. The combination may also further comprise additional therapeutic agents, not recited in the list, and/or component(s), like but not limited to pharmaceutical excipients or administration vehicles.

In a particular embodiment, the therapeutic agents may be administered simultaneously, separately, or sequentially in the treatment of a disease.

### Identification of TRIM21 as the Ligand of CLEC-1

According to the knowledge of the applicant, prior method to identify the ligand(s) of CLEC-1 allowed to identify the interaction of CLEC1 with a ligand overexpressed by necrotic tumor cells but not to identify the ligand. The applicant has developed an inventive method leading to TRIM21 as a key endogenous ligand, said method comprising the use of specific non-denaturing conditions.

CLEC-1 is part of the C-type Lectin family. The compounds belonging to this family recognize a wide variety of exogenous and endogenous ligands such as carbohydrates protein lipids dans nucleic acids. Previous attempts to identify endogenous ligands of CLEC1 revealed that at least one ligand, of proteic nature, is found in secondary necrotic cells. However, several approaches developed to identify at least a ligand of CLEC-1 did not reveal any valuable ligand.

The limitations were first due to the uncertain nature of the ligand(s). This challenge requires the use of different experimental conditions and various starting material. A technical finetuning was needed to get and maintain an interaction of a dead sensor to a potentially labile ligand released by secondary necrotic cells. The solution was elaborated after testing without success several approaches to identify CLEC-1 ligand, among which: 1) double-hybrid screening of a leucocyte/activated mononuclear cells cDNA library; 2) Ligand capture trial by a panel of diverse CLEC-1 fusion proteins immobilized on Surface Plasmon Resonance (Biacore) chips from a diversity of cellular extracts expected to contain CLEC-1 ligand(s) (MoDC, PBMC, K562 cell line) combined to a mass spectrometry identification of retained ligand; 3) immunoprecipitation of CLEC-1 on a cellular extract obtained after in cellulo CLEC-1 binding to its ligand, crosslinking and extraction of cellular protein.

Despite substantial efforts, none of these approaches led to the identification of a candidate-ligand that could be further validated by ELISA or Blitz for selective interaction with CLEC-1. There was a need of developing a sensitive and selective method to capture, isolate and identify the CLEC-1 ligand(s) known to be expressed in cells undergoing secondary necrosis and in dendritic cells.

The applicant developed an uncommon chemically mild protein extraction method, based on low detergent concentration, combined to mechanical constraints based on fast freezing and thawing to maximize cell compartments breaking to extract proteins from cells while maintaining their post-translational modifications.

A focus was made on cells in which CLEC-1 binding after membrane integrity loss (e.g. RAJI cell line) was observed. To maximize the CLEC-1 binding to its endogenous ligand, while maintaining a low nonspecific binding, the applicant sets up an immunoprecipitation method including:
1) direct binding of Fc-CLEC-1 or Fc-control to Protein G beads; without using any intermediate antibody;
2) use of magnetic beads to minimize nonspecific binding;
3) interaction in PBS only with no additional detergent except the low detergent concentration present in the cellular extract;
4) mild washing with no detergent, increasing stringency to remove unspecific bound contaminants only with high salt concentration (advantageously 500 mM NaCl)
5) elution of bound ligands with DTT denaturation only at room temperature, to avoid protein, and especially glycoprotein aggregation due to heat.

This original method led to the isolation of discrete bands from the eluate on SDS-PAGE. The analysis by mass spectrometry resulted in identification of TRIM-21 as endogenous CLEC-1A ligand, confirmed by ELISA for its selective interaction to CLEC-1A.

### Method for Screening Antagonists and Agonists of the Binding Between CLEC-1 and TRIM21

In another aspect, the invention is directed to a method for screening compounds, including but not limited to small organic compounds, antibodies or antigen binding fragments thereof, polypeptides (in particular functional equivalent of human CLEC-1 or of human TRIM21), in particular therapeutic agents which bind to TRIM21 or to CLEC-1, and that target (i.e. disrupt, reduce, inhibit or enhance) CLEC-1/TRIM21 signaling pathway, in particular to compounds that compete with or antagonize the binding between CLEC-1 and TRIM21, which comprises:
a) Providing at least one compound and assessing its(their) capability to interfere with CLEC-1/TRIM21 signaling pathway, in particular which interfere with the interaction between CLEC1 and TRIM21, more particularly which competes with or antagonizes binding between CLEC-1 and TRIM21, wherein in particular the interaction is determined between Fc-CLEC-1 fusion molecule and at least one domain of TRIM21, in particular full length TRIM21, or between CLEC-1 and at least one domain of TRIM21, in particular full length TRIM21;
b) Measuring in presence of the compound an activity of the CLEC1/TRIM21 signaling pathway and/or binding between CLEC-1 and TRIM21,
c) when the compound(s) enhance(s) or decrease(s) the activity of the CLEC1/TRIM21 signaling pathway, and/or the binding measured in step b) as compared to a negative control, identifying the compound(s) which modulate(s) the activity of the CLEC1/TRIM21 signaling pathway and/or the binding between CLEC-1 and TRIM21, in particular identifying the compound(s) which enhance(s) or reduce(s) the interaction between CLEC-1 and TRIM21.

The measure in step b) may be assessed by measuring the phagocytosis capability of myeloid cells, in particular of dendritic cells and/or macrophages in presence of the compound and compare the results with what is observed in absence of the compound. The phagocytosis capability of myeloid cells, in particular of dendritic cells and/or macrophages may be assessed on TRIM21-positive cells, in particular on TRIM21-positive tumor cells or secondary necrotic cells, particularly on tumor cells or secondary necrotic cells.

In another aspect, the invention is directed to a method for screening compounds, including but not limited to small organic compounds, antibodies or antigen binding fragments thereof, polypeptides (in particular functional equivalent of human CLEC-1 or of human TRIM21), in particular therapeutic agents which bind to TRIM21 or CLEC-1, and that target (i.e. disrupt, reduce or inhibit) the binding between CLEC-1 and TRIM21, in particular to compounds that compete with or antagonize or agonize the binding between CLEC-1 and TRIM21, which comprises:
a) Providing at least one compound and assessing its(their) capability to interfere with the binding between CLEC-1 and TRIM21, in particular to compete with or to antagonize binding between CLEC-1 and TRIM21, in particular the interaction is determined between CLEC-1 and at least one domain of TRIM21;
b) the case being, isolating and/or identifying the compound(s) which modulate(s) the interaction (e.g. binding) between CLEC-1 and TRIM21, in particular isolating and/or identifying the compound(s) which enhance(s) or reduce(s) the interaction between CLEC-1 and TRIM21.
c) Optionally, testing the binding capability of the compound(s) which modulate(s) the interaction (e.g. binding) between CLEC-1 and TRIM21 to TRIM21, in particular to human TRIM21, and/or to CLEC-1, in particular to human CLEC-1.

The invention also relates to a method for selecting a compound useful for treating a cancer with TRIM21-positive tumor cells comprising performing said method for screening compounds as herein described, wherein the compound which decreases the activity of the CLEC1/TRIM21 signaling pathway and/or the binding between CLEC-1 and TRIM21 is selected.

In particular, a method for selecting an antagonist that binds to TRIM21, especially binds specifically to TRIM21, and which antagonize the binding between CLEC-1 and TRIM21, in particular between human CLEC-1 and human TRIM21, may comprise the following steps:
a) Providing TRIM21 protein or a portion thereof comprising at least one domain of TRIM21 selected from the group consisting of RING, B-box, coiled-coil, PRY, PRYSPRY and SPRY, and CLEC-1 protein or a portion thereof comprising the extracellular domain of CLEC-1, in particular Fc-CLEC-1, more particularly Fc-CLEC-1 comprising SEQ ID No. 3,
b) Providing a compound for which the capability of antagonizing the binding between CLEC-1 and TRIM21 is to be tested,
c) Measuring in presence of the compound the binding between CLEC-1 and TRIM21,
d) Optionally, measuring the binding of the compound to TRIM21, in particular to human TRIM21, and/or measuring the binding of the compounds to CLEC-1, in particular human CLEC-1;
   the compound being an antagonist of the binding between CLEC-1 and TRIM21 when it decreases the binding measured in step c), as compared to a negative control, the compound further specifically binding to TRIM21 when the binding to TRIM21 measured in step d) is higher than the binding to CLEC-1, in particular when the affinity of the compound for TRIM21 is at least two-fold greater to its affinity CLEC-1.

A negative control may correspond to a compound of similar nature (e.g. an antibody when the tested compound is an antibody, an organic molecule when the tested compound is an organic molecule), known for not interfering with the binding between CLEC-1 and TRIM21, for example because the control does not bind to nor interfere with one or the two molecules TRIM21 and CLEC-1.

The invention is also directed to a method for selecting a compound which binds to TRIM21, especially binds specifically to TRIM21 and which is an antagonist of the binding between CLEC-1 and TRIM21, in particular between human CLEC-1 and human TRIM21, said method comprising:
a) Providing at least one compound for testing its(their) capability to bind to TRIM21 and interfere with the interaction between CLEC-1 and TRIM21, in particular to compete with or to antagonize binding between CLEC-1 and TRIM21, wherein in particular the interaction is determined between a polypeptide or a peptide comprising the extracellular domain of CLEC-1 and at least one domain of TRIM21, in particular full length TRIM21, or between Fc-CLEC-1 fusion molecule and at least one domain of TRIM21, in particular full length TRIM21, in particular the interaction is determined between CLEC-1 and at least one domain of TRIM21, in particular full length TRIM21;
b) Measuring in presence of the compound the interaction (e.g. binding) between CLEC-1 and TRIM21,
c) when the compound(s) enhance(s) or decrease(s) the binding measured in step b) as compared to a negative control, identifying the compound(s) which modulate(s) the binding between CLEC-1 and TRIM21, in particular identifying the compound(s) which enhance(s) or reduce(s) the interaction between CLEC-1 and TRIM21.

An antagonist according to the invention, in particular an antagonist which binds to TRIM21, especially binds specifically to TRIM21 and antagonizes the binding between CLEC-1 and TRIM21 can be identified by the following method comprising at least the following steps:
a) providing a plurality of cells expressing TRIM21 or a fragment thereof comprising at least one domain of TRIM21 selected from the group consisting of RING, B-box, coiled-coil, PRY and SPRY;
b) incubating said cells, in particular a lysate of said cells, with a candidate compound for which its capability to antagonize the binding between CLEC-1 and TRIM 21 is to be tested in presence and/or in absence of a polypeptide comprising at least a portion of extracellular domain of CLEC-1; c) determining whether said candidate compound binds to and blocks, suppresses, or reduces the binding between CLEC-1 and TRIM21; and
d) selecting the candidate compound that binds to and blocks, suppresses, or reduces the binding between CLEC-1 and TRIM21.

### Method for Assessing the Likeliness of a Treatment to be Effective

In another aspect, the invention relates to a method for assessing the likelihood of effectiveness of a treatment with an anti-cancer agent and/or with an antagonist of the CLEC-1/TRIM21 signaling pathway, in particular in a human patient diagnosed with a cancer, in particular a cancer with TRIM21-positive tumor or with TRIM21-positive tumor cells, such as breast cancer, hepatocellular carcinoma, lymphoma, more particularly B-cell lymphoma, colon cancer, thyroid cancer, liver cancer, renal cancer, melanoma, colorectal cancer, nasopharyngeal carcinoma, pancreatic cancer, a chronic infection, a sepsis, an infection, in particular by a Coxsackievirus or by an encephalitis virus, more particularly by Coxsackievirus B3 or Japanese encephalitis virus, a cardiovascular disease, an auto-immune disease, in particular Sjogren's syndrome or systemic lupus erythematosus or systemic sclerosis, an inflammatory disease, in particular a cancer, said method comprising:
the determination of the expression of TRIM21 in a biological sample in particular in a biological sample comprising tumor cells, wherein the sample has been previously obtained from the human patient;
when the sample is positive for expression of TRIM21, concluding that the patient is likely to benefit from the treatment with an anti-cancer agent and/or antagonist compound according to the invention, in particular an anti-TRIM21 antagonist antibody or antigen-binding fragment thereof.

The invention also relates to a method for assessing the likelihood of effectiveness of a treatment with an anti-cancer agent and/or with a compound that is an antagonist of the CLEC-1/TRIM21 signaling pathway, like an antibody or antigen-binding fragment thereof or antigen-binding antibody mimetic as described above, a functional equivalent of CLEC-1 or TRIM21, a compound that reduce the expression of TRIM21, when the sample is positive for the expression of the human TRIM21, the administration of a therapeutic amount of an anticancer agent and/or an anti-human TRIM21 or an anti-CLEC-1, like an antibody and/or an antigen-binding fragment thereof.

The invention also concerns an in vitro or ex vivo method for determining if treating a patient having a disease, with an anti-TRIM21 in particular anti-human TRIM21 compound, an anti-CLEC-1 in particular an anti-human CLEC-1 compound or with a functional equivalent of TRIM21, in particular human TRIM21 or a functional equivalent of CLEC-1, in particular human CLEC-1, is likely to be effective, the method comprising detection of the expression of TRIM21 in a biological sample previously obtained from the patient, the effectiveness of the treatment being likely when TRIM21 is detected on the surface of cells and/or within the cytosol of cells, in particular tumor cells, present within a biological sample previously obtained from the patient

In an aspect the invention concerns an in vitro or ex vivo method for determining if treating a patient having a disease, with an anti-TRIM21 in particular anti-human TRIM21 compound, an anti-CLEC-1 in particular an anti-human CLEC-1 compound or with a functional equivalent of TRIM21, in particular human TRIM21 or a functional equivalent of CLEC-1, in particular human CLEC-1, is likely to be effective, wherein the disease is a cancer, in particular cancer with TRIM21-positive tumor cells, such as breast cancer, hepatocellular carcinoma, lymphoma, more particularly B-cell lymphoma, colon cancer, thyroid cancer, liver cancer, renal cancer, melanoma, colorectal cancer, nasopharyngeal carcinoma, pancreatic cancer, or the disease is a chronic infection, a sepsis, an infection, in particular by a Coxsackievirus or by an encephalitis virus, more particularly by Coxsackievirus B3 or Japanese encephalitis virus, a cardiovascular disease, an auto-immune disease, in particular Sjogren's syndrome or systemic lupus erythematosus or systemic sclerosis, an inflammatory disease, and wherein the method of determining if treating the patient for this disease with an antagonist compound according to the invention as mentioned above is likely to be effective, comprises detection of the expression of TRIM21 in a biological sample previously obtained from the patient, the effectiveness of the treatment being likely when TRIM21 is detected on the surface of cells and/or within the cytosol of cells, in particular tumor cells, present within a biological sample previously obtained from the patient.

In a further aspect, the invention thus relates to a method for treating a human patient diagnosed with a cancer, in particular cancer with TRIM21-positive tumor cells, such as breast cancer, hepatocellular carcinoma, lymphoma, more particularly B-cell lymphoma, colon cancer, thyroid cancer, liver cancer, renal cancer, melanoma, colorectal cancer, nasopharyngeal carcinoma, pancreatic cancer, a chronic infection, a sepsis, an infection, in particular by a Coxsackievirus or by an encephalitis virus, more particularly by Coxsackievirus B3 or Japanese encephalitis virus, a cardiovascular disease, an auto-immune disease, in particular Sjogren's syndrome or systemic lupus erythematosus or systemic sclerosis, an inflammatory disease, in particular a cancer, wherein the method comprises the steps of:
determining the expression of TRIM21 in a biological sample, in particular in a biological sample comprising tumor cells wherein the sample has been previously obtained from the human patient and,
when expression of TRIM21 is positive, administering an agent, in particular an anti-cancer agent, and/or an anti-human TRIM21 antagonist, like but not limited to an antibody or antigen-binding fragment thereof, to said human patient.

In a further aspect, the invention relates to a method for enhancing the phagocytosis capability of myeloid cells, in particular of dendritic cells and/or macrophages, in particular for enhancing the phagocytosis of TRIM21-positive cells by myeloid cells, in particular of tumor cells and/or secondary necrotic cells, particularly of TRIM21-positive tumor cells and/or TRIM21-positive secondary necrotic cells, the method comprising:
selecting a patient having a disorder associated with TRIM21-positive cells, in particular a disorder associated with TRIM21-positive tumor cells and/or TRIM21-positive secondary necrotic cells; and
administering to the patient an effective amount of the compound of the invention, which inhibits the CLEC-1/TRIM21 signaling pathway.

In a further aspect, the invention relates to a method for enhancing the phagocytosis capability of myeloid cells, in particular of dendritic cells and/or macrophages, in particular for enhancing the phagocytosis of TRIM21-positive cells by myeloid cells, in particular of tumor cells and/or secondary necrotic cells, particularly of TRIM21-positive tumor cells and/or TRIM21-positive secondary necrotic cells, the method comprising the administration to a patient in need thereof of an effective amount of a compound of the invention, which inhibits the CLEC-1/TRIM21 signaling pathway.

In a further aspect, the invention relates to the use of a compound that inhibits the C-type lectin-like receptor-1 (CLEC-1)/tripartite motif-containing protein 21 (TRIM21) signaling pathway, in particular that antagonizes the binding between CLEC-1 and TRIM21, more particularly between human CLEC-1 and human TRIM21, wherein the compound binds to TRIM21, especially binds specifically to TRIM21, or to an intracellular molecule involved in the CLEC-1/TRIM21 signaling pathway or reduces the expression of functional TRIM21 or is a functional equivalent of TRIM21, in particular an antagonist compound of the binding between CLEC-1 and TRIM21 that binds to TRIM21, more particularly binds to human TRIM21, especially binds specifically to TRIM21, for the manufacture of a medicament for the treatment of a patient having a disease, in particular a disease selected from the group consisting of a cancer, in particular a cancer with TRIM21-positive tumor cells, such as breast cancer, hepatocellular carcinoma, lymphoma, more particularly B-cell lymphoma, colon cancer, thyroid cancer, liver cancer, renal cancer, melanoma, colorectal cancer, nasopharyngeal carcinoma, pancreatic cancer, a chronic infection, a sepsis, an infection, in particular by a Coxsackievirus or by an encephalitis virus, more particularly by Coxsackievirus B3 or Japanese encephalitis virus, a cardiovascular disease, an auto-immune disease, in particular Sjogren's syndrome or systemic lupus erythematosus or systemic sclerosis, an inflammatory disease.

In particular, the method for treating a human patient diagnosed with cancer, in particular cancers with TRIM21-positive tumor cells, such as breast cancer, hepatocellular carcinoma, lymphoma, more particularly B-cell lymphoma, colon cancer, thyroid cancer, liver cancer, renal cancer, melanoma, colorectal cancer, nasopharyngeal carcinoma, pancreatic cancer, a chronic infection, a sepsis, an infection, in particular by a Coxsackievirus or by an encephalitis virus, more particularly by Coxsackievirus B3 or Japanese encephalitis virus, a cardiovascular disease, an auto-immune disease, in particular Sjogren's syndrome or systemic lupus erythematosus or systemic sclerosis, an inflammatory disease, in particular a cancer, may comprise, prior to administration of the agent, in particular the anti-cancer agent, or during the treatment with such an agent, in particular such an anti-cancer agent, or after a treatment with a first or a further agent, in particular anti-cancer agent, a step of determining whether a biological sample obtained from the patient, in particular a biological sample comprising diseased cells, in particular tumor cells, comprises cells expressing TRIM21. When said sample may be regarded as being TRIM21 positive, particularly when patient has TRIM21-positive tumor cells, treatment is administered.

The determination of the expression of human TRIM21 may involve the use of a chimeric molecule comprising the extracellular domain of CLEC-1 as described herein, in particular the use of Fc-CLEC-1 as described herein.

The compounds of the invention may be administered as a therapeutic agent, in particular as an anti-cancer agent, or may be added as a further active agent in a treatment with another agent, in particular an anti-cancer agent, in particular to a patient having CLEC-1 positive tumor cells and/or with TRIM21 positive tumor cells.

Accordingly, the invention provides TRIM21 expressed in cells of a biological sample previously taken from a patient such as a sample comprising tumor cells or apoptotic cells or necrotic cells, for use as a positive marker for the selection of patients for the treatment with immunotherapeutic anti-cancer agents or with chemotherapeutic anti-cancer agents. Optionally said use comprises the quantification of the marker.

In a further aspect, the invention thus relates to a method for treating a human patient diagnosed with a cancer, the human patient having a TRIM21-positive tumor. A TRIM21-positive tumor may correspond to a tumor wherein at least one cell expresses TRIM21. The invention thus also concerns a method wherein the presence of TRIM21 or abnormal expression of TRIM21 within tumor cells is measured in a biological sample (previously) obtained from the patient, and when the tumor cells express TRIM21 or exhibit abnormal expression of TRIM-21, a compound that inhibits the CLEC-1/TRIM21 signaling pathway as detailed herein is administered to the patient. Abnormal expression of TRIM21 can be assessed by comparing the expression of TRIM-21 is a healthy cell obtained from the same patient, or a healthy cell obtained from another patient.

The invention also relates to the use of TRIM21, in particular human TRIM21, as a biomarker in an in vitro test for assessing if treating a patient having a disease, in particular a cancer, more particularly a cancer with TRIM21-positive tumor cells, with a compound binding to CLEC-1 or TRIM21, which is an antagonist of the binding between CLEC-1 and TRIM21, is likely to be effective.

Further aspects and features of the invention will be found in the following examples and in the figures.

The following Figures and Examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

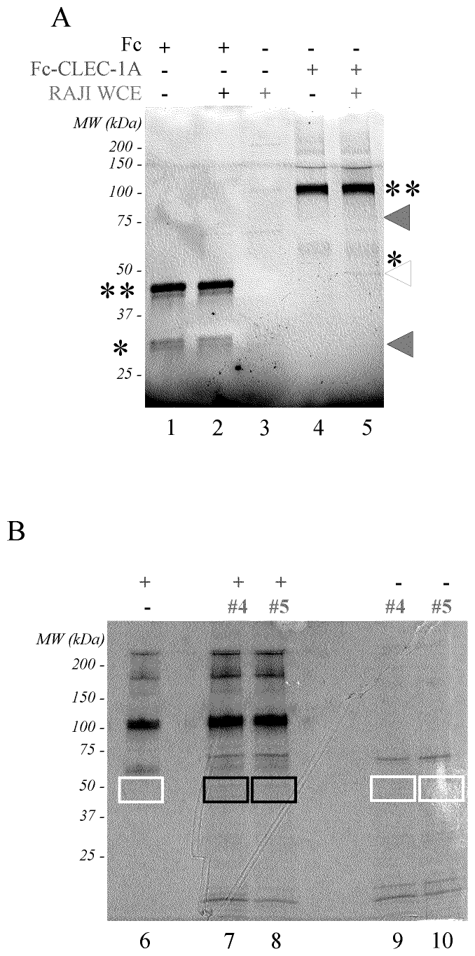

FIG. 1: SDS-PAGE of the immunoprecipitation of Fc-CLEC-1 with the content of RAJI cell line lysate. A: Immunoprecipitation elution products were separated by SDS-PAGE on a stain free 4-15% gel. Protein G magnetic beads were coated with Fc only (lanes 1 and 2); no Fc-protein (lane 3); Fc-CLEC-1 protein (lanes 4 and 5). Fc-CLEC-1A immunoprecipitation of RAJI WCE (Whole Cell Extract) proteins (line 5) was compared to several controls: Fc only (lane 1); Fc+RAJI WCE (lane 2), RAJI WCE only (lane 3), and Fc-CLEC-1A only (lane 4). Grey and white triangles indicate discrete bands only present in the Fc-CLEC-1A+RAJI WCE condition (lane 5). Asterisks indicate monomeric * (i.e. partially denatured) and dimeric ** Fc-fused recombinant proteins. B: Immunoprecipitation elution products were separated by SDS-PAGE on a 4-15% gel stained with colloidal coomassie. Immunoprecipitation was performed as in A. The squares indicate the area cut from the gel for mass spectrometry analysis, corresponding to major bands specific to Fc-CLEC-1-RAJI WCE co-immunoprecipitation (squares in lanes 7 and 8 correspond to white triangle in A, and issued from the same extract as lane 5). Squares in lanes 6 and 9 and 10 correspond to the controls at the apparent same size level (lane 6: Fc-CLEC only; lanes 9 and 10: RAJI WCE only).

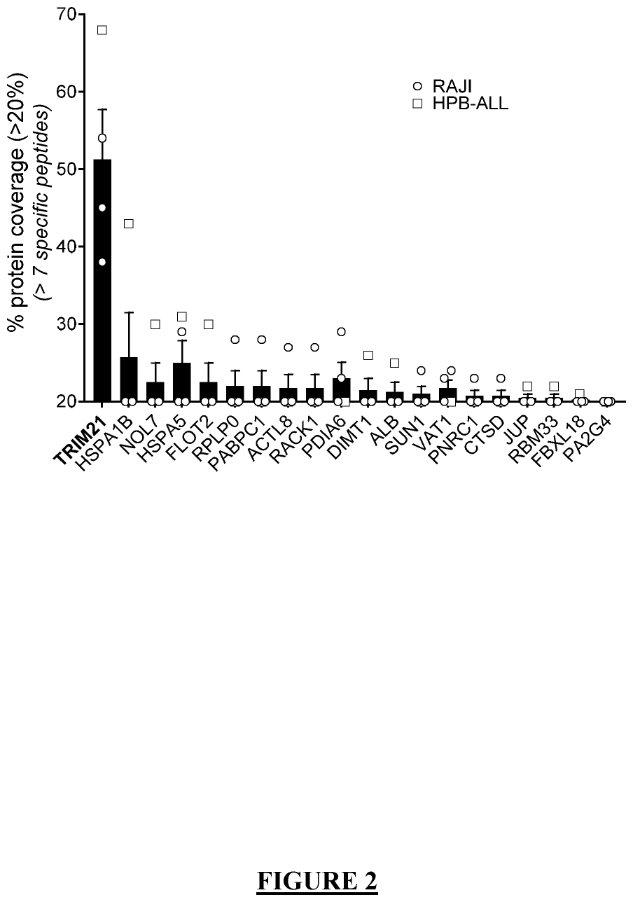

FIG. 2: Analysis of the ligand candidates for CLEC-1A. Molecular analysis of the content of the 50 kDa band extracted from SDS-PAGE illustrated on FIG. 1 and of the total eluates obtained after cellular protein extracts interaction with beads bound with Fc-CLEC-1. RAJI corresponds to extract issued from RAJI cells. HPB-ALL corresponds to extracts issued from leukemic cell line referenced HPB-ALL (DSMZ No. ACC 483).

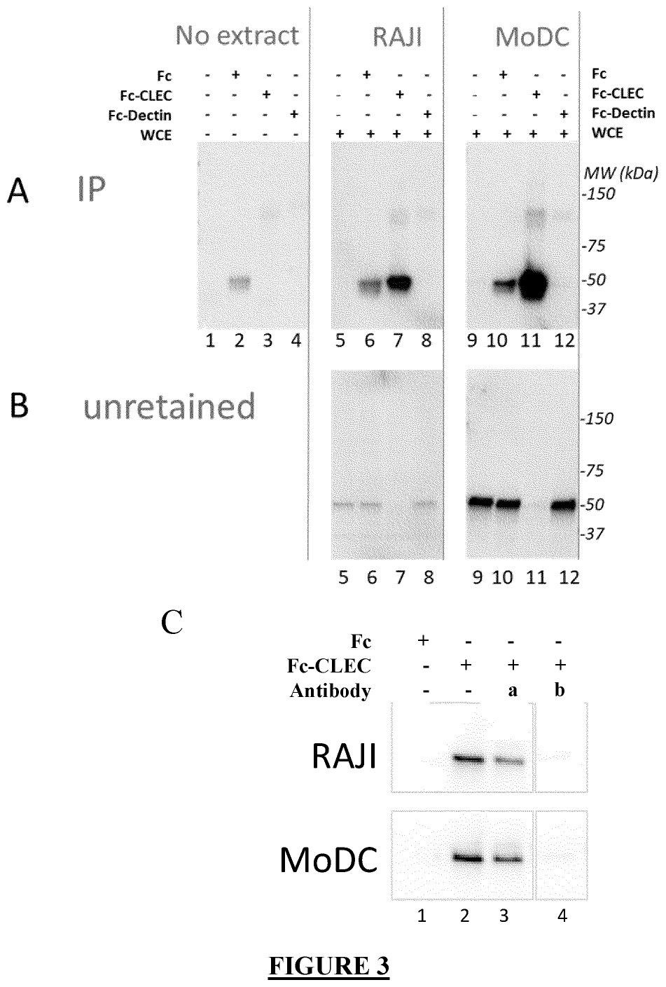

FIG. 3: Co-immunoprecipitation of TRIM21 with CLEC-1A. Immunoprecipitation of WCE proteins from RAJI cells (lanes 5 to 8) or from MoDC (lanes 9 to 12) were performed with magnetic beads bearing Fc-only (lanes 2, 6 and 10), Fc-CLEC-1A (lanes 3, 7 and 11), Fc-dectine-1A (lanes 4, 8 and 12) or no Fc-fusion protein (lane 1, and 9. Lanes 1 to 4 are controls made without cell extract. A: Western Blot revelation of co-immunoprecipitated (IP) proteins with an anti-TRIM21 antibody. TRIM 21 is revealed (band at 52 kDa) in the samples with Fc-CLEC-1A. B: Western blot of unretained extracts (samples depleted from IP supernatant). Fc-CLEC-1a is also depleted (lines 7 and 11). C: A co-immunoprecipitation of RAJI (top) or MoDC (bottom) Whole Cell Extract was performed with immobilized Fc-CLEC in presence of a 50 fold molar excess of diverse antibodies: A. negative control antibody, B. anti-TRIM21 antibody (PA5-22294, Invitrogen). Bound TRIM21 was revealed by Western Blot.

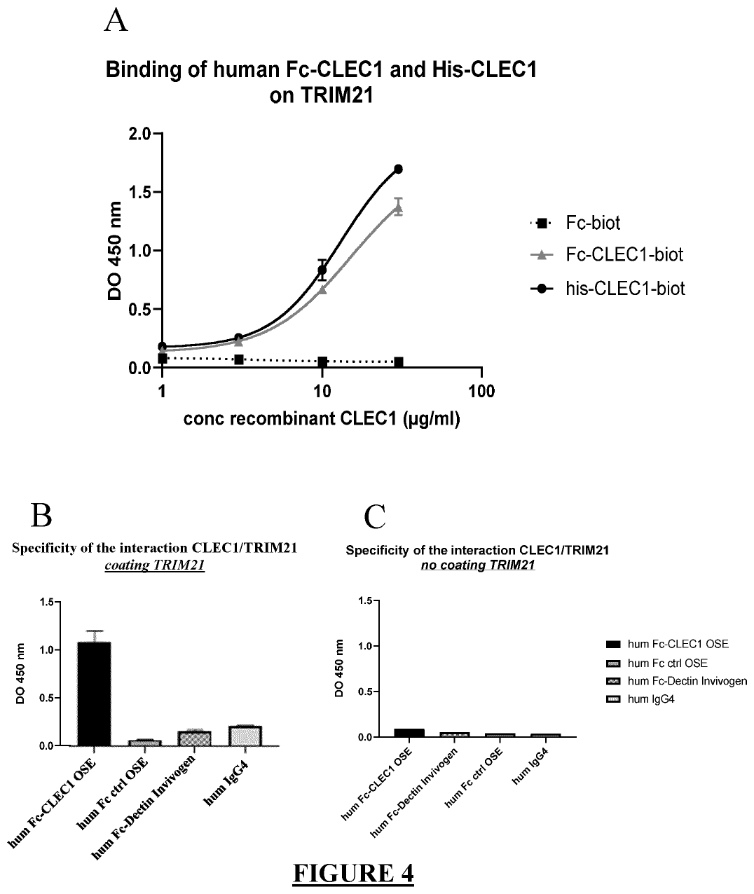

FIG. 4. ELISA binding of human Fc-CLEC1 and His-CLEC1 on TRIM21. Recombinant human TRIM21 protein was immobilized on a plate. Plate was saturated and a dose response of biotinylated Fc-CLEC1-A proteins was captured by TRIM21. Fc-CLEC1-biot and His-CLEC1-biot corresponds to the CLEC1 protein as described herein, either with a His-tag or a Fc portion, and which are biotinylated. Hum Fc ctrl OSE corresponds to a home-made human Fc receptor domain.

FIG. 5: ELISA measurement of antagonistic activity of anti-TRIM21 antibodies on the binding between CLEC-1 and TRIM21. (A) localization of the epitopes targeted by anti-TRIM21 antibodies within the full-length TRIM21 protein. Boxes represent different domains of TRIM21, and the arrows represent the regions recognized by the antibody associated with each arrow. Numbers correspond to the amino acid residue localization within full-length TRIM21 (B) The anti-TRIM21 antibodies used in the experiment are listed on the right of the graph. Recombinant human TRIM21 protein was immobilized on a plate. Plate was saturated with the anti-TRIM21 antibodies and a dose response of biotinylated Fc-CLEC1 protein.

FIG. 6: Interaction between TRIM21 and CLEC-1 in presence of different anti-CLEC1-antibodies (αCLEC-1 mAb #3 and αCLEC-1 mAb #5), an anti-TRIM21 antibody (Fisher PA5-22295) and a control antibody that does not bind to CLEC-1 nor TRIM21. (A) represents the percentage of interaction between TRIM21 and CLEC1 as compared to a base line corresponding to the experiment in presence of the control antibody. (B) is a SDS-PAGE illustrating the co-immunoprecipitation of TRIM21 with Fc-CLEC or Fc-control in presence of the above-mentioned antibodies (C) BIACORE.

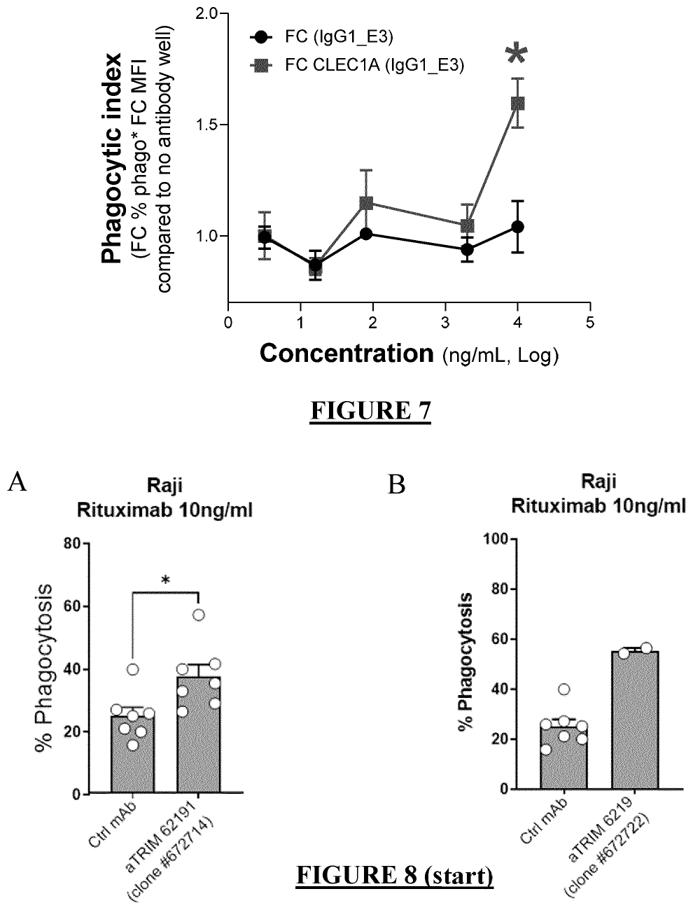

FIG. 7: Phagocytosis index of HPB-ALL cells by human macrophages in presence of increasing concentrations of FC-CLEC-1A or Fc-control peptide, as compared to untreated HPC-ALL cells.

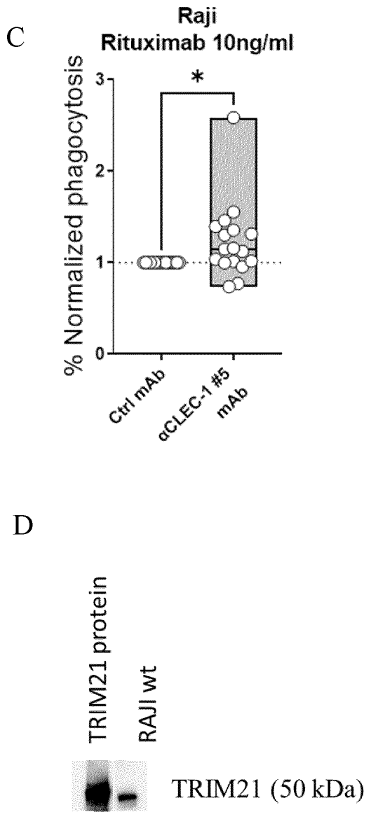

FIG. 8: Phagocytosis of TRIM-21 positive tumor cells (Raii cells) in presence of Rituximab (at 10 ng/ml) and different anti-TRIM21 antibodies or anti-CLEC1-antibody or a control antibody. (A), (B) and (C). Anti-TRIM21 antibodies corresponds to anti-TRIM21 antibodies referenced MAB-62191 (FIG. 8A) MAB-6219(1) (FIG. 8B) in the present description which binds to the amino acid sequences localized between amino acid residues 195 and 293 of TRIM21 of SEQ ID No. 2 or to the immunogen corresponding to amino acid sequence set forth in SEQ ID No. 8. Anti-CLEC1 antibody corresponds to anti-CLEC1 antibody referenced αCLEC-1 mAb #5 having the heavy chain of SEQ ID No. 10 and the light chain of SEQ ID No. (FIG. 8C) in the present description. (D): Western Blot anti-TRIM21 on Raji cell lysate. Raji cell lysate (50 μg sample) and control recombinant TRIM21 protein (0.010 μg) were migrated on SDS-Page stain-free gel 10% (Biorad), transferred on nitrocellulose membrane and incubated with anti-TRIM21 antibody (clone AB01/1G5 (Biorad, #VMA00730)) before to be revealed with peroxidase labelled anti-mouse antibody (Jackson Immunoresearch #715-036-151). TRIM21 protein was detected at 50 KDa on Raji lysate and control protein.

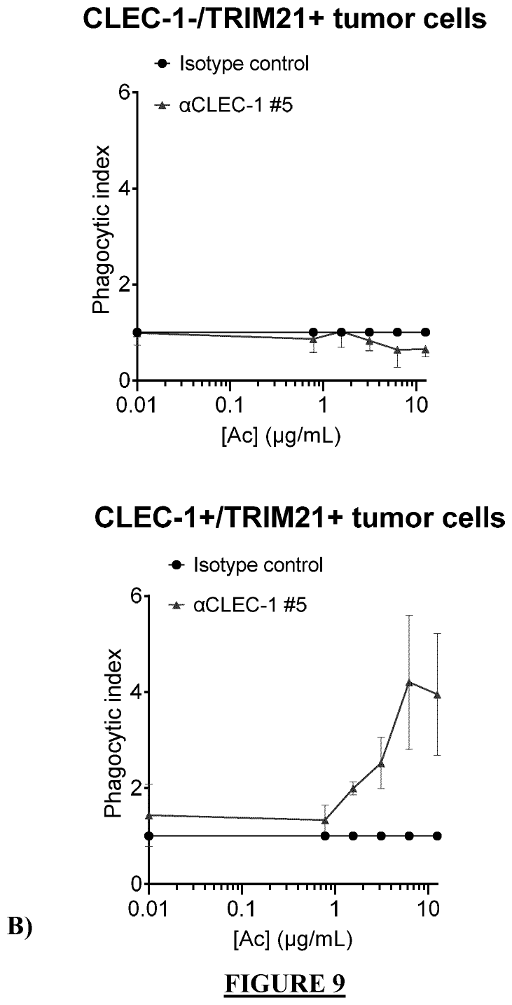

FIG. 9: Positive effect of an antagonist monoclonal antibody inhibiting CLEC-1 interaction with TRIM21 on macrophage-mediated phagocytosis. A: Macrophage-mediated phagocytic activity is not enhanced by αCLEC-1 mAb #5 treatment inhibiting CLEC-1 interaction with TRIM21, in the context of a tumor cell line lacking CLEC-1 expression (but expressing TRIM21). B: Enhancement of macrophage-mediated phagocytic activity by treatment with αCLEC-1 #5 inhibiting CLEC-1 interaction with TRIM21 in the context of a tumor cell line expressing both CLEC-1 and TRIM21.

### MATERIAL AND METHODS

### Production of Recombinant Soluble Human Fc-CLEC1 Protein

Soluble Clec1-human IgG Fc chimera (Fc-Clec-1) was generated by cloning into pcDNA3.1 the extracellular domain of human Clec-1 (Q74-D280) fused in N-terminal to human IgG1e3 Fc sequence. IgG1e3 Fc sequence contains the following mutations E233P/L234V/L235A/ΔG236+A327G/A330S/P331S (substitutions to IgG2 and IgG4 residues to reduce FcγRI binding and ADCC/CDC). The detailed sequence is available in the technical datasheet pFuse-hIgG1e3-Fc1 (Invivogen #pfc1-hg1e3). The synthesis of Fc-Clec sequence was ordered at Genscript with restriction site Xhol in 5′ and Xbal in 3′ for cloning in pcDNA3.1. For expression in mammalian cells Kozak sequence (GCCACC—SEQ ID No.10) and IgK leader signal peptide (METDTLLLWVLLLWVPGSTGD—SEQ ID No.9) were added in the beginning of the sequence. Midiprep of plasmid pcDNA3.1-Fc-Clec was produced with Nucleobond Xtra Midi EF kit (Macherey Nagel #740420.50). Recombinant Fc-Clec protein was produced in Hek free style cells (Thermofisher #R79007) by transiently transfection using 293 Fectine transfection reagent (Thermofisher #12347019). 60.10⁶ Hek free style cells were transfected with 60 μg of plasmid pcDNA3.1-Fc-Clec and 120 μl of 293 fectine in 60 ml of free style 293 medium (Thermofisher #12338018). The production was maintained 5 days, then supernatant was clarified by centrifugation 30 min at 3000G and filtered on 0.22 μM Stericup (Merck Millipore #SCGPU05RE). The clarified supernatant was purified on HiTrap protein A column (GE #17040201) with an elution in 0.1M citric acid pH3. Eluate was concentrated on Microsep Advance 10 kDa (Pall #MCP010C41) and filtered on 0.22 μM filter.

### Induction of Monocyte Derived Dendritic Cells (MoDC)

MoDC were generated from monocytes obtained after magnetic PBMC sorting with a Classical monocytes kit Miltenyi (130-117-337) or CD14 beads (130-050-201).

Monocytes underwent differentiation for 6-7 days in culture in complete RPMI medium (10% FCS; 1% PS; 1% L-Gln; 1×Na pyruvate; 1×NEAA; 1 mM HEPES) supplemented with 50 ng/mL GM-CSF (Miltenyi-130-095-372) and 20 ng/ml IL-4 (Miltenyi-130-093-917). The immature Dendritic Cells (iDC) were further brought to fully differentiated MoDC by 2 days of culture in presence of 20 ng/ml TGFb.

Production of Whole Cell Protein Extracts (WCE) from Cells

The lymphoblastic cell line RAJI and MoDC cells were extensively washed with cold PBS. Then cell pellet was resuspend in 5 volumes of ice cold mild RIPA-IP lysis buffer composed of PBS (Corning), 1% NP-40, 0.25% Na deoxycholate, 1× Protease Inhibitor cocktail (PIC) (all from Sigma Aldrich). To complete the cell lysis, the samples were subjected to rapid freezing at −150° C. followed by thawing in ice cold water. The cycle was repeated twice. The cell lysates were clarified by centrifugation at 14000g for 15 minutes at 4° C. The proteic concentration was assessed by BCA assay.

### Fc-Fusion Protein Immunoprecipitation and Candidate Band Isolation

Protein G Surebeads magnetic beads (Biorad) were prepared following the manufacturer's recommendations and resuspent in PBS+0.1% Tween-20 (Sigma Aldrich). 30 pmol of Fc-fusion protein (Fc only, Fc-CLEC-1, or Fc-hDectin-1A (Invivogen) were added to an original volume of 30 ul beads per IP. The Fc-fusion proteins were allowed to bound to the beads for 1h on a turning wheel at +4° C. After 3 washes in PBS-tween followed by one wash in PBS, 1 mg of WCE extract was added, in a final volume of 600 ul of cold PBS+1×PIC. The interaction was performed for 2-3 h at +4° C. on the turning wheel. The supernatant was collected and the magnetic beads were subjected to 5 minutes washes in 600 ul on the wheel followed by 2 minutes of magnetic capture, all at +4° C.: once in PBS, twice in PBS containing 500 mM NaCl, twice in PBS. The bound proteins were recovered after a 10 min incubation at room temperature in 15 ul of elution buffer composed of 1× Laemmli buffer (BioRad) containing 250 mM DTT (Sigma). The eluate was collected and proteins separated on SDS-PAGE. SDS-polyacrylamide gels were stained with colloidal Coomassie: 0.08% Coomassie brilliant blue G250, 10% citric acid, 8% ammonium sulfate (all from Sigma), 20% Methanol (Fisher) for 6h to overnight under slow shaking. Gels were destained with distilled water until the background was clear. Bands of interested were cut out and studied by mass spectrometry analysis.

### In Gel Digestion, Extraction and Mass Spectrometry Analysis

After bands extrusion, the gel plugs were washed four times with 50 μL of 25 mM ammonium hydrogen carbonate (NH4HCO3) and 50 μL of acetonitrile. The cysteine residues were reduced by 50 μL of 10 mM dithiothreitol at 57° C. and alkylated by 50 μL of 55 mM iodoacetamide. After dehydration with acetonitrile, the proteins were cleaved in gel with 200 ng of modified porcine trypsin (Promega) in 25 mM NH4HCO3. The digestion was performed overnight at 37° C. The generated peptides were extracted with 60% acetonitrile in 0.1% formic acid followed by a second extraction with 100% acetonitrile. Acetonitrile was evaporated under vacuum and the peptides were resuspended in 25 μL of H20 and 0.1% formic acid prior to mass spectrometry analysis. NanoLC-MS/MS analyses of the tryptic peptides were performed on a nanoACQUITY Ultra-Performance_LC-system (Waters, Milford, MA) coupled to a TripleTOF 5600 mass spectrometer (ABSciex) equipped with a nanoelectrospray ion source.

The samples were trapped on a Symmetry C18 precolumn (20×0.18 mm, 5 μm, Waters Corp.) and the peptides were separated on a ACQUITY UPLC® BEH130 C18 column (75 μm×250 mm, 1.7 μm particle size, Waters Corp). The solvent system consisted of 0.1% formic acid in water (solvent A) and 0.1% formic acid in acetonitrile (solvent B). Trapping was performed during 3 min at 5 μL/min with 99% of solvent A and 1% of solvent B. Elution was performed at a flow rate of 300 nL/min, using 3-25% gradient (solvent B) over 60 min, followed by 25-40% over 20 min, then 80% (solvent B) over 10 min at 60° C.

The mass spectrometer was operated in positive mode, with the following settings: ion spray voltage floating (ISVF) 2300 V, curtain gas (CUR) 25 psi, interface heater temperature (IHT) 75° C., ion source gas 1 (GS1) 2 psi, declustering potential (DP) 100 V. Information-dependent acquisition (IDA) mode was used with top 5 MS/MS scans. The MS scan had an accumulation time of 50 ms on m/z [400-1250] range and the MS/MS scans 50 ms m/z [150-1600] range in high sensitivity mode. Switching criteria were set to ions with charge state of 2-4 and an abundance threshold of more than 150 counts, exclusion time was set at 12 s. IDA rolling collision energy script was used for automatically adapting the CE. Mass calibration of the analyser was achieved using peptides from digested BSA. The complete system was fully controlled by AnalystTF 1.6 (AB Sciex).

Raw data collected were processed and converted in “.mgf peak list” format.

### Data Analysis and Protein Identification

Search engine MASCOT 2.6.2 algorithm (Matrix Science) was used. The search was performed against the Human database concatenated with the same reversed sequences (“target/decoy” database). Trypsin was selected as cleavage enzyme and a maximum of one missed cleavage was allowed, one variable modification (oxidation of methionine residues), one fixed modification (carbamidomethylation of cysteines). Spectra were searched with a mass tolerance of 20 ppm for MS and 0.07 Da for MS/MS data.

Mascot results were loaded into the Proline software for the validation (http://proline.profiproteomics.fr). For validation a false discovery rate (FDR) of 1% was applied to the protein set (at the protein level).

### Western Blotting

After proteins separated by SDS-PAGE were transferred on nitrocellulose membranes, blocking was completed in PBS-tween+5% BSA (Sigma). The membranes were successively incubated with anti-TRIM-21 (SCBT sc-25351 1/1000e) O/N at 4° C. and anti-mouse-HRP conjugate (Jackson) and revealed with Clarity ECL substrate (Biorad)

### ELISA

Measurement of Fc-CLEC-1A and TRIM21 interactions were performed by ELISA.Polysorp 96 well plates (Nunc) were coated with 50 μl per well of 2 μg/ml TRIM21-his (Sino 18010-H07B) in carbonate buffer (pH 9.2), O/N at 4° C. Plate was saturated with superblock buffer (#37515, Thermofisher). Fc and Fc-CLEC-1A biot coupled proteins were captured at a concentration of 30 μg/ml in 50 μl PBS-Tween 0.05%-BSA 1% for 2 h at 37° C. After extensive washes, bound proteins were detected with 1/1000 diluted Streptavidin-PO (JI 016-030-084) in PBS-Tween 0.05%, 50 μl/well for 1 h at 37° C. and TMB (Sigma T8665) is used for revelation.

Antagonistic effects of commercial anti-TRIM21 antibodies on CLEC1/TRIM21 binding interaction was measured by ELISA. Recombinant human TRIM21 protein (#18010-H07B, Sinobiological) was immobilized on 96 well Polysorp plate at 2 μg/ml in carbonate buffer (pH 9.2), O/N at 4° C. Plate was saturated with superblock buffer (#37515, Thermofisher) and commercial anti-TRIM21 antibodies were added in excess (20 μg/ml). Biotinylated human Fc-CLEC1 (#07/08/20, OSE IMMUNO) or His-CLEC1 (#1704-CL, Biotechne) proteins were added at 30 μg/ml in dose response to measure binding. After incubation and washing, streptavidin-peroxidase (#016-030-084, Jackson Immunoresearch, USA) was added and revealed by conventional methods.

### CLEC/TRIM21 Interaction on BIAcore

Analysis was performed on T200 (Cytiva, France) by Platform PP2I (Montpellier, France). Recombinant human TRIM21 protein (produced in Baculovirus, from Sinobio #18010-H07B) was immobilized into a CM5 sensor chip (Cytiva) at 20 μg/ml in acetate 10 mM pH5 buffer. His-CLEC (produced in NSO cell line, from Biotechne #1704-CL) was injected at 1 μM alone or after co-incubation with antagonistic anti-CLEC mAb at 5 μM on immobilized TRIM-21 recombinant protein.

### Antagonistic Assay by Co-IP

Co-IP were performed like in the Fc-fusion protein immunoprecipitation method described above, with adjusted quantities and the following changes: 5 pmol of Fc-fusion proteins, 150 ug WCE and 250 pmol of antibodies tested for their antagonistic activity were used in each reaction. The antibodies were incubated for 30 minutes at +4° C. with their targets (WCE) for anti-TRIM antibodies; beads bound Fc-CLEC fusion protein for anti-CLEC antibodies) before the loading of the extract on the beads bearing Fc-CLEC fusion protein. The eluted proteins were separated by SDS-PAGE and revealed by Western blotting. The quantification was performed through the Chemidoc Imaging system (BioRad).

### Phagocytosis Assays

In vitro phagocytosis assays were performed by 1-hour coculture of 2.5×104 human M1 macrophages labeled with CellTrackerGreen (ThermoFisher, Waltham, Massachusetts, USA, 1/2000, 20 min at 37° C.) and 5×104 leukemic HPB-ALL cells or Raji cells labeled with CPD (ThermoFisher, 1/2000 10 min at 37° C.) in serum-free RPMI. Phagocytosis was analyzed by a CytoFLEX flow cytometer (Beckman, Brea, California, USA) and analysis using FlowJo software (TreeStar, BD Life Sciences Franklin Lakes, New Jersey, USAs). The phagocytic index was calculated as follows: fold change of percentage of CPD+ cells in CTG+ macrophages compared to the one detected in control wells (no treatment) multiplied by the fold change in geometric mean in APC fluorescence (CPD) in CTG+ macrophages compared to the one detected in control wells (no treatment). Treatments applied were Fc control (IgG1-E3) or FcCLEC1A (IgG1-E3) at the concentrations shown in FIG. 9 or anti-TRIM21 antibodies referenced MAB-6219(1) and MAB-62191 or anti-CLEC1-antibody referenced αCLEC-1 mAb #5 at a concentration of 10 μg/ml (FIG. 8). Anti-CLEC1-antibody referenced αCLEC-1 mAb #5 has the heavy chain variable domain of SEQ ID No. 10 and the light variable domain of SEQ ID No. 11.

Primary human macrophage-mediated phagocytosis of TRIM21+/CLEC1+versus TRIM21+/CLEC1− tumor cells. In vitro phagocytosis assays were performed by 15 min coculture of 2.5×10⁴ human M1 macrophages labeled with CellTrackerGreen (ThermoFisher, Waltham, Massachusetts, USA, 1/2000, 20 min at 37° C.) and 5×10⁴ tumor cells labeled with CPD (ThermoFisher, 1/2000 10 min at 37° C.) in serum-free RPMI. Tumor cells used in these assays were THP1 cells expressing TRIM21 but not CLEC-1 or THP1 cells expressing both TRIM21 and CLEC-1 (CLEC-1 expression introduced by lentiviral transduction of the human full-length sequence of CLEC-1 gene). Phagocytosis was analyzed by a CytoFLEX flow cytometer (Beckman, Brea, California, USA) and analysis using FlowJo software (TreeStar, BD Life Sciences Franklin Lakes, New Jersey, USAs). The phagocytic index was calculated as follows: fold change of percentage of CPD+ cells in CTG+ macrophages compared to the one detected by treatment with istotype control multiplied by the fold change in geometric mean in APC fluorescence (CPD) in CTG+ macrophages compared to the one detected with isotype control. The antagonist monoclonal antibody used in this assay FIG. 9 is αCLEC-1 mAb #5 and corresponding isotype control (Evitria, Schlieren, Switzerland).

### Example 1: Identification of TRIM21 as a Ligand of CLEC-1

Previous attempts to capture CLEC-1A ligands from libraries or from endogenous cellular proteins obtained under strong ionic detergent conditions did not succeed. It has been shown that CLEC-1A ligand was found abundantly in permeabilized cells from the RAJI cell line. As these cells are easily expandable in cell culture, a large amount of cellular protein extract in which the ligand would be available could be prepared. As previous attempts using strong detergents did not allow the capture of a yet abundant intracellular ligand, a concern was the quality and integrity of the ligand extracted from the cells. In order to obtain such integrity, the applicant has searched specific appropriated conditions, focusing on milder detergent extraction and interaction conditions.

RAJI cells were lysed in RIPA-IP buffer with low anionic detergent content and subjected to mechanical cell compartments disruption by repeated freeze-thaw cycles. To identify the ligands of CLEC-1 from the resulting whole cell extracts (WCE), fusion proteins composed of a dimeric form of human CLEC1A extracellular domains fused to the Fc portion of human IgG were used. The CLEC7a (Dectin-1) fusion proteins and the dimeric Fc fragments were used as controls. First, Fc, Fc-CLEC1A or no capture protein were directly bound to protein G magnetic Surebeads through their Fc fragment. The RAJI WCE was submitted to interaction with these beads bound proteins, carefully washed in low detergent but high salt conditions and eluted with 250 mM DTT without heating. A SDS-PAGE separation of the eluted proteins revealed a clear background in the control conditions (FIG. 1A, lanes 1 to 4) with only few bands, specific of Fc-CLEC-1A (FIG. 1A, lane 5). The low background confirms our hypothesis that mild interaction conditions were needed to capture the ligand without denaturing it. The experiment was repeated in duplicate to cut out the Fc-CLEC1A major specific band around 50 kDa for mass spectrometry analysis (FIG. 1B).

The mass spectrometry analysis was performed on this band or on the total eluate obtained after the interaction of RAJI or HPB-ALL WCE with beads bound Fc-CLEC vs Fc only. The raw data were filtered to eliminate classical contaminants (e.g. skin proteins) and proteins that bound to Fc as well. The final short list contains proteins that were identified with at least 7 specific peptides (FIG. 2). TRIM21 E3 Ubiquitin-protein Ligase was outstanding from this short list, being the only one identified with over 50% sequence coverage in triplicate conditions on RAJI cells and simplicate conditions on HPB-ALL cells. The other candidates had lower scores and were mainly cytoskeletal and abundant cellular proteins.

### Example 2: Interaction Between TRIM21 and CLEC-1

As TRIM21 is known to be an intracellular Fc Receptor, it was crucial to verify that the binding to Fc-CLEC-1A was mediated through CLEC-1A itself and not its Fc moiety. Immunoprecipitation was thus repeated, using Fc and Fc-Dectin as controls, and revealed the bound proteins by and anti-TRIM21 Western Blot. With RAJI cell extracts, this confirmed that TRIM21 from a RAJI WCE was strongly co-immunoprecipitating (co-IP) with Fc-CLEC-1A (FIG. 3A, lane 7) while the signal was undetectable with Fc-Dectin (lane 8) and much lower with Fc-only (lane 6). This weak signal was indeed mainly due to the background signal given by Fc only in absence of cellular extract, probably due to cross-reactive detection of Fc by the secondary HRP-conjugated anti-mouse Fc antibody. The specific interaction between Fc-CLEC-1A and TRIM21 was further sustained by the fact that the supernatant after the IP-reaction, containing the unretained proteins (FIG. 3B) was depleted of TRIM21 only by Fc-CLEC-1A bearing magnetic beads and not by the beads bearing Fc only, Fc-Dectin or no Fc-fused protein (compare lane 7 to lanes 5, 6 and 8). The experiment was repeated using Monocyte derived Dendritic Cells (MoDC) protein extracts as starting endogenous protein mix. The same specific coIP of TRIM21 was observed (FIG. 3A lane 11), even with a stronger interaction signal and a very clear depletion of TRIM21 from the protein extract by CLEC-1A (FIG. 3B lane 11). Altogether, these experiments prove a strong and specific interaction between Fc-CLEC-1A and endogenous TRIM21 that is not mediated through the Fc part of the fusion protein, but through CLEC-1A itself. The reality of this interaction is sustained by the fact that we have observed CLEC-1A binding to permeabilized RAJI cells and to MoDC before. On FIG. 3C, it is illustrated that anti-TRIM21 antibody prevented the binding of TRIM21 to immobilized CLEC-1 (line 4) as compared to the negative control antibody (line 3), thus displaying an antagonistic effect on the CLEC-1/TRIM21 interaction of the anti-TRIM21 antibody.

The co-immunoprecipitation reveals an interaction between CLEC-1A and endogenous TRIM21 in a cellular protein mix. However, to prove a direct interaction without any partner that could intervene in the cellular extract, ELISA experiments were performed with recombinant Fc-CLEC1A, His-CLEC1A and TRIM21 proteins. An ELISA dose-response curve of Fc-coupled or His-coupled proteins binding on TRIM21 revealed a selective binding of CLEC-1A to TRIM21 (FIG. 4A), starting at a concentration of 1 μg/ml and no binding with any of the Fc-fused protein controls. Interestingly, there was no binding to Fc alone (Fc-biot control), suggesting that at these concentrations, the interaction between CLEC-1A and TRIM21 was not mediated through the Fc fragment. Furthermore, no interaction was observed between TRIM21 and the other C-type lectin receptor Dectine-1 (FIG. 4B), supporting a specific and selective TRIM21-CLEC-1A direct interaction. Human His-CLEC1 and human Fc-CLEC1 bind human TRIM21 with an EC50 of respectively 13 and 15 μg/ml. This is further illustrated on FIG. 4B, wherein at a fixed concentration of 10 μg/ml, (CLEC1A or controls), Fc-CLEC1A is the sole protein interacting with the coated TRIM21 proteins. No unspecific binding of either Fc-fusion proteins was observed in absence of coated TRIM21. As illustrated on FIG. 4C, Fc-CLEC-1 at a concentration of 10 μg/ml binds on a surface coated with TRIM21 and is not able to bind on the surface uncoated with TRIM21. It is therefore clear that TRIM21 and CLEC-1 interact. Therefore, compounds which bind TRIM21 or CLEC1A and are able to antagonize the interaction between these two molecules represent potent therapeutic agents, in particular to treat disease wherein TRIM21 and/or CLEC-1, particularly TRIM21/CLEC-1 signaling pathway, are involved. This ELISA interaction validation confirms the co-IP results and validate the direct interaction of endogenous TRIM21 from lymphoma cells or dendritic cells with CLEC-1A, thus identifying the first endogenous CLEC-1A ligand. To sum up, biotinylated His-CLEC-1A and FC-CLEC1A bind to TRIM21. The binding between CLEC-1 and TRIM21 is unrelated to the Fc domain and these experiments illustrate a direct interaction between CLEC-1 and TRIM21. To further illustrate this direct interaction between CLEC-1 and TRIM21, biotinylated Fc-CLEC-1 and His-CLEC-1 has been added on a surface coated or not with human TRIM21.

### Example 3: Antagonist Compounds of the Binding Between CLEC-1 and TRIM21

Five commercial anti-TRIM21 antibodies were tested for their antagonistic activity on TRIM21/CLEC1 interaction.

| Manu- | | | | Immunogen |
|---|---|---|---|---|
| facturer | Reference | Isotype | Clonality | (amino acid) |
| Santacruz | sc-25351 | Mouse IgG1 | Monoclonal | 141-280 |
| Fisher | PA5-22294 | Rabbit IgG | Polyclonal | 254-457 |
| Fisher | PA5-18147 | Goat IgG | Polyclonal | C terminus |
| | | | | of TRIM21 |
| Biotechne | MAB6219 | Mouse IgG1 | Monoclonal | 195-293 |
| Biotechne | MAB62191 | Mouse IgG2a | Monoclonal | 195-293 |

Anti-TRIM21 antagonist antibodies sc-25351, PA5-22294, PA5-18147 and MAB-62191, MAB-621911 and are particularly promising antagonizing towards the interaction between CLEC1A and TRIM21. On FIG. 5A are localized the domains of interaction between each anti-TRIM21 antibody used in the present examples and TRIM21 protein. As observed on FIG. 5B, the interaction between Fc-CLEC1 and TRIM21 is decreased by more than 50% by these antibodies. Thus, several and different anti-TRIM21 antibodies antagonize specifically the binding between CLEC-1 and TRIM21.

Anti-CLEC-1 and anti-TRIM21 antagonist antibodies were also tested to assess their respective effects on the TRIM21/CLEC-1 interaction in corresponding assays. In these experiments, the antagonistic effect of two in-house anti-CLEC-1 antibodies (αCLEC-1 mAb #3 and #5) and the anti-TRIM21 antibody Fisher PA5-22295 were compared; these three antibodies reduce the binding between CLEC-1 and TRIM21 by 40%, 55% and 65% respectively (FIG. 6A). The same effect was measured in an antagonist assay on a co-IP of cellular endogenous TRIM-21 by Fc-CLEC (FIG. 6B). These results illustrate that anti-TRIM21 antibodies may be useful for reducing or inhibiting the interaction between CLEC-1 and TRIM21.

### Example 4: Phagocytosis of TRIM21-Positive Cells by Myeloid Cells in Presence of a Compound that Blocks the CLEC-1/TRIM21 Signaling Pathway

Illustrated on FIG. 7 are results of phagocytosis of HPB-ALL cells, a model of T cell leukemia, in presence of Fc-CLEC1A, a compound that disrupt the binding between CLEC-1A and TRIM21. The inventors found that treatment of the T-ALL cell line HPB-ALL, which are TRIM-21 positive cells, with FcCLEC1A, thereby preventing the interaction of CLEC1A with its ligand TRIM21, induces an increase in the phagocytic activity of human macrophages towards HPB-ALL cells, whereas this phenomenon is not detected upon treatment of tumor cells with an Fc control.

On FIG. 8, results obtained in a combination therapy on TRIM21-positive tumor cells (Raji cells) with Rituximab and different anti-TRIM21 antibodies (FIG. 8A-B) or anti-CLEC-1 antibody (FIG. 8C), which are all antagonist of the binding between CLEC-1 and TRIM21, are illustrated. As illustrated on FIG. 8D Raji cells are TRIM21-positive tumor cells. When the two types of antibodies are administered, the phagocytosis of TRIM21-positive tumor cells (RAJI cells) is of approximately 40% of total TRIM21-positive tumor cells (Raji cells), as compared to a phagocytosis percentage of 25% when the anti-TRIM21 antibody is absent. Thus, the phagocytosis of TRIM21-positive tumor cells (RAJI cells) is greatly improved when the CLEC-1/TRIM21 signaling pathway is disturbed.

FIG. 9 illustrates the positive effect of an anti-CLEC-1 antibody, which is an antagonist of the binding between CLEC-1 and TRIM21, on macrophage-mediated phagocytosis. Macrophage-mediated phagocytic activity was not enhanced by said anti-CLEC-1 antibody, in the context of a tumor cell line lacking CLEC-1 expression (but expressing TRIM21). However, anti-CLEC-1 antibody, which is an antagonist of the binding between CLEC-1 and TRIM21, enhanced the macrophage-mediated phagocytic activity in the context of a tumor cell line expressing both CLEC-1 and TRIM21. All of these data provide a functional application for the inhibition of the interaction of CLEC1A with its ligand TRIM 21.

1-30. (canceled)

31. A compound that is an antagonist of the binding between CLEC-1 and TRIM21, in particular an antagonist of the binding between human CLEC-1 and human TRIM21, for use in the treatment of a human subject suffering from a cancer with TRIM21 positive tumor cells.

32. The compound according to claim 31 for use according to claim 31 which binds to CLEC-1, in particular to human CLEC-1 or which is a polypeptide that is a functional equivalent of CLEC-1, in particular of human CLEC-1.

33. The compound according to claim 31 or 32 for use according to claim 31 or 32 wherein the compound inhibits the CLEC-1/TRIM21 signaling pathway, and wherein the compound binds to CLEC1, in particular to human CLEC-1 or reduces the expression of functional CLEC1 or is a polypeptide which is a functional equivalent of CLEC1.

34. The compound according to claim 32 or 33 for use according to claim 32 or 33, which binds to CLEC-1, in particular to human CLEC-1, more particularly to the extracellular domain of human CLEC-1 and which is an antibody, an antigen-binding fragment thereof or an antigen-binding antibody mimetic.

35. The compound according to claim 34 for use according to claim 34, which is a chimeric antibody, a humanized antibody, a recombinant antibody, a fully-humanized antibody, a monoclonal antibody, a de-immunized antibody, in particular a humanized monoclonal antibody.

36. The compound according to claim 34 or 35, for use according to claim 34 or 35, which competes for the binding to CLEC-1, in particular to human CLEC-1, with an anti-CLEC-1A antibody having the heavy chain variable domain of SEQ ID No. 10 and the light chain variable domain of SEQ ID No. 11.

37. The compound according to claim 32 or 33 for use according to claim 32 or 33, which is a polypeptide that is a functional equivalent of human CLEC-1, in particular this polypeptide is fused to an immunoglobulin constant domain.

38. The compound according to any one of claims 31 to 37 for use according to claim 31 to 37, which increases the phagocytosis capability of myeloid cells, in particular dendritic cells and/or macrophages, in particular which increases the phagocytosis of TRIM21-positive cells, more particularly of TRIM21-positive tumor cells and/or secondary necrotic cells, by dendritic cells and/or macrophages, in particular which increases the phagocytosis of tumor cells and/or secondary necrotic cells by dendritic cells and/or macrophages.

39. The compound according to any one of claims 31 to 38, for use according to any one of claims 31 to 38, by increasing the phagocytosis of TRIM 21-positive tumor cells.

40. The compound according to claim 31 or 32, for use according to claim 31 or 32, which binds to TRIM21, in particular to human TRIM21, or which is a polypeptide that is a functional equivalent of TRIM21, in particular of human TRIM21.

41. The compound according to claim 40 for use according to claim 40, which increases the phagocytosis capability of myeloid cells, in particular dendritic cells and/or macrophages, in particular which increases the phagocytosis of TRIM21-positive cells by dendritic cells and/or macrophages, in particular which increases the phagocytosis of tumor cells and/or secondary necrotic cells, more particularly of TRIM21-positive tumor cells and/or TRIM21-positive secondary necrotic cells, by dendritic cells and/or macrophages.

42. The compound according to claim 40 or 41 for use according to claim 40 or 41, wherein the subject suffers from a cancer with TRIM21-positive tumor cells.

43. The compound according to any one of claims 31 to 42, for use according to any one of claims 31 to 42, wherein the human subject suffers from a cancer with TRIM21 positive tumor cells and CLEC-1 positive tumor cells.

44. The compound according to any one of claims 41 to 43 for use according to any one of claims 41 to 43, which is an antibody, an antigen-binding fragment thereof, an antigen-binding antibody mimetic, or a peptide or a polypeptide which is a functional equivalent of TRIM21, in particular this polypeptide is fused to an immunoglobulin constant domain.

45. The compound according to any one of claims 41 to 44, for use according to any one of claims 41 to 44, which binds to an epitope sequence localized within the coiled-coil domain of TRIM21, in particular the coiled-coil domain localized between amino acid residues 128 and 238 of TRIM21 of SEQ ID No. 2, and/or which binds to an epitope sequence localized within the PRY-SPRY domain of TRIM21, in particular the PRY-SPRY domain localized between amino acid residues 268 and 465 of TRIM21 of SEQ ID No. 2.

46. The compound according to any one of claims 41 to 45 for use according to any one of claims 41 to 45, which is a chimeric antibody, a humanized antibody, a recombinant antibody, a fully-humanized antibody, a monoclonal antibody, a de-immunized antibody, in particular a humanized monoclonal antibody.

47. The compound according to any one of claims 41 to 46, for the use according to any one of claims 41 to 46, which is an antibody, an antigen-binding fragment thereof or an antigen-binding antibody mimetic that competes with at least one competing antibody selected among the list consisting of anti-TRIM21 antibodies sc-25351, PA5-22294, PA5-18147 and MAB-62191 for the binding to TRIM21, in particular for the binding to contiguous amino acid residues localized within the coiled-coil domain of TRIM21, in particular the coiled-coil domain localized between amino acid residues 128 and 238 of TRIM21 of SEQ ID No. 2, and/or for the binding to contiguous amino acid residues localized within the PRYSPRY domain of TRIM21, in particular the PRYSPRY domain localized between amino acid residues 268 and 465 of TRIM21 of SEQ ID No. 2, and wherein the antibody, antigen-binding fragment thereof or antigen-binding antibody mimetic enhances the phagocytosis capability of myeloid cells, in particular dendritic cells and/or macrophages, in particular which increases the phagocytosis of TRIM21-positive cells, more particularly of TRIM21-positive tumor cells and/or secondary necrotic cells, by dendritic cells and/or macrophages, in particular which increases the phagocytosis of tumor cells and/or secondary necrotic cells by dendritic cells and/or macrophages.

48. The compound according to any one of claims 41 to 47, for use according to any one of claims 41 to 47, by increasing the phagocytosis of TRIM 21-positive cells.

49. A combination of compounds comprising a first therapeutic agent and at least one further therapeutic agent, wherein:
a) The first therapeutic agent is a compound as defined according to any one of claims 31 to 48; and
b) at least one of the further therapeutic agent selected from the list consisting of a tumor-targeting antibody or antigen-binding fragment thereof, in particular a tumor-targeting monoclonal antibody or antigen-binding fragment thereof, a tumor-targeting monoclonal antibody or antigen-binding fragment thereof which activates and/or enhances the phagocytosis of tumor cells by myeloid cells, especially by macrophages, or more particularly a monoclonal antibody selected from the group consisting of alemtuzumab, atezolizumab, bevacizumab, cetuximab, herceptin, panitumumab, rituximab, trastuzumab, an anti-PDL-1 antibody and an anti-CD47 antibody, and/or another antibody or monoclonal antibody selected from the group consisting of an anti-PD1 antibody and an anti-SIRPa antibody; and/or a chemotherapeutic agent, in particular a cytotoxic agent with anti-proliferative, pro-apoptotic, cell cycle arresting and/or differentiation inducing effect, more particularly a cytotoxic agent selected from the group consisting of cytotoxic antibody, alkylating drugs, anthracyclines, antimetabolites, anti-microtubule agents, topoisomerase inhibitors, alkaloids, bleomycin, antineoplastic drugs, cyclophosphamide, for simultaneous, separate or sequential use in the treatment of a human subject suffering from a cancer with TRIM21 positive tumor cells.

50. The combination of compounds according to claim 49, wherein the first therapeutic agent is selected from the group consisting of an antibody, an antigen-binding fragment thereof, an antigen-binding antibody mimetic which binds to TRIM21, or a peptide or a polypeptide which is a functional equivalent of TRIM21, in particular of human TRIM21.

51. The combination of compounds according to claim 49, wherein the first therapeutic agent is selected from the group consisting of an antibody, an antigen-binding fragment thereof, an antigen-binding antibody mimetic which binds to CLEC-1, or a peptide or a polypeptide which is a functional equivalent of CLEC-1, in particular of human CLEC-1.

52. The combination of compounds according to claim 41, wherein the first therapeutic agent is selected from the group consisting of an antibody, an antigen-binding fragment thereof, an antigen-binding antibody mimetic which binds to CLEC-1 and which competes for the binding to CLEC-1, in particular to human CLEC-1, with an anti-CLEC-1A antibody having the heavy chain variable domain of SEQ ID No. 10 and the light chain variable domain of SEQ ID No. 11.

53. The combination of compounds according to claim 41, wherein the first therapeutic agent is a polypeptide which is a functional equivalent of CLEC-1, in particular this polypeptide is fused with an immunoglobulin constant domain.

54. A method for selecting a compound which modulates, in particular which enhances or reduces, more particularly which inhibits, the interaction between C-type lectin-like receptor-1 (CLEC-1) and tripartite motif-containing protein 21 (TRIM21), in particular a compound that is an antagonist of the binding between CLEC-1 and TRIM21, in particular between human CLEC-1 and human TRIM21, said method comprising:
a) Providing at least one compound and assessing its(their) capability to interfere with the interaction between CLEC1 and TRIM21, more particularly which competes with or antagonizes binding between CLEC-1 and TRIM21, in particular the interaction is determined between CLEC-1 and at least one domain of TRIM21;
b) Measuring in presence of the compound the binding between CLEC-1 and TRIM21,
c) when the compound(s) enhance(s) or decrease(s) the binding between CLEC1 and TRIM21 measured in step b) as compared to a negative control, identifying the compound(s) which modulate(s) the interaction between CLEC-1 and TRIM21, in particular the compound(s) which enhance(s) or reduce(s) the interaction between CLEC-1 and TRIM21.

55. A method for selecting a compound useful for treating a cancer with TRIM21-positive tumor cells comprising performing the method as defined in claim 54 wherein the compound which modulates the interaction between CLEC1 and TRIM21, in particular the binding between CLEC-1 and TRIM21 is selected.


SEQUENCE LISTING (11 sequences)

SEQ ID NO: 1 - protein, 280 aa, synthetic construct
  REGION 1..280: note = Synthetic sequence
MQAKYSSTRDMLDDDGDTTMSLHSQGSATTRHPEPRRTEHRAPSSTWRPVALTLLTLCLVLLIGLAALGLLFFQYYQLSNTGQDTISQMEERLGNTSQELQSLQVQNIKLAGSLQHVAEKLCRELYNKAGAHRCSPCTEQWKWHGDNCYQFYKDSKSWEDCKYFCLSENSTMLKINKQEDLEFAASQSYSEFFYSYWTGLLRPDSGKAWLWMDGTPFTSELFHIIIDVTSPRSRDCVAILNGMIFSKDCKELKRCVCERRAGMVKPESLHVPPETLGEGD

SEQ ID NO: 2 - protein, 475 aa, synthetic construct [sequence omitted]

SEQ ID NO: 3 - protein, 207 aa, synthetic construct
  REGION 1..207: note = Synthetic sequence
QYYQLSNTGQDTISQMEERLGNTSQELQSLQVQNIKLAGSLQHVAEKLCRELYNKAGAHRCSPCTEQWKWHGDNCYQFYKDSKSWEDCKYFCLSENSTMLKINKQEDLEFAASQSYSEFFYSYWTGLLRPDSGKAWLWMDGTPFTSELFHIIIDVTSPRSRDCVAILNGMIFSKDCKELKRCVCERRAGMVKPESLHVPPETLGEGD

SEQ ID NO: 4 - protein, 111 aa, synthetic construct
  REGION 1..111: note = Synthetic sequence
LEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRCHS

SEQ ID NO: 5 - protein, 198 aa, synthetic construct
  REGION 1..198: note = Synthetic sequence
ELRSVCHVPGLKKMLRTCAVHITLDPDTANPWLILSEDRRQVRLGDTQQSIPGNEERFDSYPMVLGAQHFHSGKHYWEVDVTGKEAWDLGVCRDSVRRKGHFLLSSKSGFWTIWLWNKQKYEAGTYPQTPLHLQVPPCQVGIFLDYEAGMVSFYNITDHGSLIYSFSECAFTGPLRPFFSPGFNDGGKNTAPLTLCPL

SEQ ID NO: 6 - protein, 221 aa, synthetic construct
  REGION 1..221: note = Synthetic sequence
ESWNLKDLDITSPELRSVCHVPGLKKMLRTCAVHITLDPDTANPWLILSEDRRQVRLGDTQQSIPGNEERFDSYPMVLGAQHFHSGKHYWEVDVTGKEAWDLGVCRDSVRRKGHFLLSSKSGFWTIWLWNKQKYEAGTYPQTPLHLQVPPCQVGIFLDYEAGMVSFYNITDHGSLIYSFSECAFTGPLRPFFSPGFNDGGKNTAPLTLCPLNIGSQGSTDY

SEQ ID NO: 7 - protein, 139 aa, synthetic construct
  REGION 1..139: note = Synthetic sequence
VALGELRRKQELAEKLEVEIAIKRADWKKTVETQKSRIHAEFVQQKNFLVEEEQRQLQELEKDEREQLRILGEKEAKLAQQSQALQELISELDRRCHSSALELLQEVIIVLERSESWNLKDLDITSPELRSVCHVPGLK

SEQ ID NO: 8 - protein, 99 aa, synthetic construct
  REGION 1..99: note = Synthetic sequence
RQLQELEKDEREQLRILGEKEAKLAQQSQALQELISELDRRCHSSALELLQEVIIVLERSESWNLKDLDITSPELRSVCHVPGLKKMLRTCAVHITLDP

SEQ ID NO: 9 - protein, 13 aa, synthetic construct
  REGION 1..13: note = Synthetic sequence
CPLNIGSQGSTDY

SEQ ID NO: 10 - protein, 117 aa, synthetic construct
  REGION 1..117: note = Synthetic sequence
QVQLQQSGPELVKPGASVKMSCKASGFTFTDYVIAWVKVRTGQGLEWIGEIYPGSGSIYYNEKFKGKATLTADKSSNTAYMQLSSLTSEDSAVYFCASSTVVAFDYWGQGTTLTVSS

SEQ ID NO: 11 - protein, 108 aa, synthetic construct
  REGION 1..108: note = Synthetic sequence
EIVLTQSPTTLAASPGEKIIITCSDSSSISSNYLHWYQQKPGFSPKLLIYGTSNLASGVPARFSGSGSGTTYSLTIGTMEAEDVATYYCQQGSSIPRTFGGGTKLEIK